(19) United States
(12) Patent Application Publication
(10) Pub. No.: US 2026/0215710 A1
(43) Pub. Date: Jul. 30, 2026

(54) WEARABLE DEVICES FOR NONINVASIVE RESPIRATORY QUOTIENT MONITORING VIA TRANSCUTANEOUS SENSORS AND MEASUREMENTS

(71) Applicant: RQ ADVANTAGE LLC, GAINESVILLE, FL (US)

(72) Inventor: Darwin Ang, Gainesville, FL (US)

(21) Appl. No.: 19/462,503

(22) Filed: Jan. 28, 2026

Related U.S. Application Data
(60) Provisional application No. 63/750,296, filed on Jan. 28, 2025.

(51) Int. Cl. A61B 5/1455; A61B 5/00

(52) U.S. Cl. CPC A61B 5/14552; A61B 5/4866; A61B 5/681; A61B 5/7271; A61B 2560/0462; A61B 2562/0233

(57) ABSTRACT

The disclosure provides a wrist band including a gas-sensing module including a luminescent sensor for transcutaneous measurements of carbon dioxide. The wrist band may further include a luminescent sensor for transcutaneous measurements of oxygen. The wrist band may include a luminescent sensor including 8-Hydroxypyrene-1,3,6-trisulfonic acid (HPTS). The wrist band may include a luminescent sensor including a ruthenium or platinum-based dye. The wrist band may include a luminescent sensor integrated into the wrist band as an add on strap module, in a smartwatch housing, or as a patch or armband. The wrist band may be a wrist band for a smart watch. In embodiments, the disclosure also includes methods of measuring Respiratory Quotient (RQ) using the wristband.

### RELATED APPLICATIONS

This application claims priority to U.S. Provisional Application No. 63/750,296 filed on Jan. 28, 2025, which is herein incorporated by reference in its entirety.

### BACKGROUND

### Technical Field of the Invention

The disclosure is directed to wearable devices for health management, weight management, fitness and exercise applications and including energy expenditure tracking.

### Description of the Related Art

Energy expenditure tracking is central to weight management, fitness, and overall health. Many users rely on existing fitness wearables to estimate calories burned; however, most such devices rely primarily on indirect proxies (e.g., heart rate and motion) that can produce inconsistent or inaccurate calorie estimates.

The core drawback is that current wearables cannot directly measure how the body is metabolizing nutrients in real time. Instead, they rely on approximation formulas derived from user profiles (age, weight, sex), heart rate, or motion. Consequently, they only provide rough estimates of energy expenditure (EE), making it difficult for individuals to make precise decisions about nutrition and exercise. This frustrates both casual users (who want to lose weight or stay healthy) and professional athletes/medical researchers (who need accurate, real-time physiological data).

### SUMMARY DISCLOSURE OF THE INVENTION

The disclosure provides a wrist band comprising: a strap configured to be worn around a wrist; and a gas-sensing module coupled to the strap on a user-contact side such that, during wear, the gas-sensing module contacts skin at a measurement site, the gas-sensing module comprising: a gas-permeable membrane configured to be positioned adjacent the skin; a luminescent sensing element responsive to carbon dioxide and optically coupled to the gas-permeable membrane; an excitation light source configured to illuminate the luminescent sensing element; a photodetector configured to detect emission from the luminescent sensing element; and at least one processor operatively coupled to the excitation light source and the photodetector and configured to determine a transcutaneous carbon dioxide partial pressure based on the detected emission.

The disclosure provides a wrist band including a gas-sensing module including a luminescent sensor for transcutaneously measuring carbon dioxide. The wrist band may further include a luminescent sensor for transcutaneously measuring oxygen. The wrist band may include a luminescent sensor including 8-Hydroxypyrene-1,3,6-trisulfonic acid (HPTS). The wrist band may include a luminescent sensor including a ruthenium or platinum-based dye. The wrist band may include a luminescent sensor integrated into the wrist band as an add on strap module, in a smartwatch housing, or as a patch or armband. The wrist band may be a wrist band for a smart watch. In embodiments, the disclosure also includes methods of measuring Respiratory Quotient (RQ) using the wristband.

The present disclosure is directed to wearable devices, systems, and methods for fitness and health applications. In embodiments, a wearable measures energy expenditure and respiratory quotient using transcutaneous measurements of carbon dioxide (CO₂) and optionally oxygen (O₂). In embodiments, luminescence-based sensor films can be used for CO₂ and O₂ (non-limiting examples include pH-mediated CO₂ indicators and oxygen-quenchable luminophores). In embodiments, the wearable is implemented as a strap/band, a sidecar module, a watch-like housing, a patch, or another wearable form factor that maintains stable skin contact.

In embodiments, the product is a replacement smartwatch strap (and/or an add-on strap module) that contains a noninvasive gas-sensing module on the underside of the strap. When worn, the module contacts the skin and optically measures transcutaneous CO2 (and optionally O2) using luminescent sensing films. A key measurement approach is time-domain dual lifetime referencing (t-DLR), which is a pulsed optical technique that is less sensitive to changes in excitation intensity (e.g., LED output drift, skin optical coupling changes) than pure intensity measurements.

Replaceable sensing elements may be implemented as a solid sensing disk/pad or as a membrane-sealed pocket containing particulate or microsphere-based sensing media. Consumables can be swapped on a periodic basis to address hygiene, maintain signal quality, and manage finite chemistry lifetime.

In various embodiments, the sensing module is integrated into (i) a replacement strap/band, (ii) an add on strap module (“sidecar”), (iii) a standalone smartwatch housing, (iv) a patch/armband, or (v) other wearable carriers. In embodiments that leverage an existing watch ecosystem, the strap/band is configured to attach to a host smartwatch (optionally via adapters or interchangeable connectors) and to exchange data with the host smartwatch and/or a companion device via one or more communication interfaces, including wireless and/or wired interfaces. Processing may be performed on the strap/band, on the host smartwatch, on the companion device, and/or in a cloud service, in any combination.

The strap communicates with a host smartwatch and/or a companion device (e.g., a smartphone) and may use context signals (e.g., heart rate, temperature, motion/activity) to estimate two primary metabolic outputs: Respiratory quotient (RQ)—a fuel utilization indicator (fat vs carbohydrate) related to VCO2/VO2.

Other features and aspects will be apparent from the following detailed description, the drawings, and the claims.

### BRIEF DESCRIPTION OF THE DRAWINGS

FIG. 1 shows a General Schematic Diagram of RQ Fitness Wearable.

FIG. 2 shows a Detailed Schematic Diagram of RQ Fitness Wearable.

FIG. 3 shows an RQ Fitness Wearable Technical Architecture and Potential Use Cases.

FIG. 4 shows a Comprehensive RQ Wearable Architecture: Sensors, Use Cases, and Key Considerations.

FIG. 5 shows an exemplary cross sectional diagram of a transcutaneous gas-sensing interface of the disclosure.

FIG. 6 shows an embodiment of t-DLR CO2 measurement.

FIG. 7 shows an example time-domain dual lifetime referencing (t-DLR) measurement cycle.

FIG. 8 shows an example of a smartwatch embodiment of the disclosure showing sensor 801.

FIG. 9 shows an embodiment of a sensing interface cross-section.

FIG. 10 shows another embodiment of a sensing interface cross-section.

FIG. 11 shows example processing pipeline and electronics blocks.

FIG. 12 shows example user interface layouts including an example “metabolic map” visualization that combines an RQ-derived fuel metric with an EE-derived burn metric, optionally with trend and confidence indicators.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DISCLOSURE OF THE INVENTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

### MODES FOR CARRYING OUT THE INVENTION

Respiratory Quotient (RQ), sometimes called the Respiratory Exchange Ratio (RER), is a well-established measure of metabolic fuel usage: RQ=VCO2/VO2; A lower RQ (around 0.7) typically indicates fat metabolism; An RQ near 1.0 indicates carbohydrate metabolism. Values in between reflect a mixed fuel source. Values above 1.0 are often associated with anaerobic or high-intensity exercise.

Measuring both CO₂ production and O₂ consumption transcutaneously (through the skin), allows the direct calculation of RQ. Once RQ is known, more accurate energy expenditure (EE) data can be derived in real time-leading to far more precise fitness and nutrition insights than what is currently available.

Health and Fitness Market. Casual users aiming to lose weight or maintain a healthy lifestyle can finally see how many calories they're truly burning- and whether they're burning carbs or fat. Athletes and sports enthusiasts can tailor training sessions with direct feedback on their metabolic pathways and know exactly when to replenish carbs (e.g., endurance athletes).

Medical & Clinical Applications. Patients who must follow strict dietary regimens (e.g., ketogenic diets, pre-operative nutrition) can be monitored in real time for actual metabolic changes. Elderly or malnourished patients: monitoring RQ can detect potential undernutrition or starvation states (RQ<0.7 at rest). Epilepsy patients on a ketogenic diet: verifying persistent ketosis (RQ ~0.6-0.7) can be beneficial for seizure control.

Commercial wearables are widely adopted, and there remains an unmet need for a practical, continuous, noninvasive respiratory quotient and energy expenditure monitor that improves upon proxy-only estimates. (Market sizing and acquisition examples are omitted as non-essential to patent enablement.)

Transcutaneous CO₂/O₂. Traditional “indirect calorimetry” devices require a mask or metabolic cart, which is bulky and not practical for daily use. However, newer luminescence-based sensor films—HPTS-based for CO₂ and ruthenium- or platinum-based for O₂—allow partial pressures to be measured optically through the skin.

Hardware Overview. Luminescent Sensor Films/Dyes. CO₂ film: Typically, HPTS-based (8-Hydroxypyrene-1,3,6-trisulfonic acid (HPTS)) or other pH-sensitive dyes that change fluorescence with changing CO₂ partial pressure. O₂ film: Can be ruthenium or platinum-based dyes that alter phosphorescence lifetime under different O₂ concentrations.

Light Emitters and Photodiodes. LEDs matched to sensor film excitation wavelengths (e.g., 405-470 nm for CO₂, ~450 nm for O₂). A reflective PPG LED (in embodiments, green at 530 nm) to measure heart rate and to detect respiratory wave signals from baseline drift.

Microcontroller and Wireless Module (e.g., BLE). Collects optical signals from photodiodes. Runs embedded algorithms to convert luminescence signals into partial pressures and calculates RQ. Transmits real-time data to a smartphone or cloud for further processing and display. Integrates data from the accelerometer to enable a built-in pedometer feature.

Mechanics and Form Factor. A watch-like or patch-style wearable that ensures stable skin contact. Gas-permeable membranes to allow CO₂/O₂ diffusion without letting in excess moisture. An internal or external accelerometer module can be positioned to optimize step detection.

Power and Battery. Low-power microcontroller with BLE connectivity. Duty cycles for LEDs minimized to extend battery life. Includes power considerations for the pedometer function, which can be duty-cycled or run continuously depending on user settings.

Software & Algorithms. Luminescence Phase/Intensity Calculations. Transcutaneous PaCO₂ and PaO₂ are computed via phase-based or radiometric methods that track how sensor dyes fluoresce under LED excitation. Requires on-board calibration or references.

PPG-Based Heart Rate & Respiratory Rate. Heart Rate (HR): The AC component of the PPG signal is extracted using bandpass filtering (typical range ~0.5-4 Hz). Respiratory Rate (RR): The DC or very low-frequency component of the PPG (0.1-0.4 Hz) can indicate respiratory waves. Optionally, an accelerometer can help separate true breathing signals from motion artifacts.

RQ Computation. RQ=VCO2/VO2 is estimated from partial pressures of CO₂ and O₂ (adjusted for known physiological relationships). A regression or calibration model can refine these estimates using heart rate, temperature, age, and other variables.

The energy expenditure (EE) can be computed using a Weir equation or a modified Weir equation based on estimated VO2 and VCO2 and/or related features.

Pedometer (Step Counting). An onboard accelerometer provides step detection, allowing the device to function as a pedometer. An onboard accelerometer provides step detection, allowing the device to function as a pedometer. Using this in combination with RQ data, HR, and time of day, we can make meaningful interpretation of RQ trends throughout the day of the user. See below for further explanation. It is included for user familiarity. Users can view daily step counts in parallel with the more accurate RQ-based energy expenditure data.

Trend Over Single Snapshot: Interpreting RQ in isolation (e.g., 0.7 vs. 1.0) can lead to confusion. Monitoring how RQ moves throughout the day, combined with pedometer/accelerometer data and time-of-day context, offers a much richer understanding of metabolic status.

Multiple Causes for the Same RQ: RQ>1.0 can reflect either anaerobic exercise or excessive carbohydrate intake. RQ<0.7 can reflect either a successful ketogenic fast or an unhealthy starvation state.

Contextual Feedback: Asking the user simple questions or using additional signals (step count, HR, time of day) ensures the wearable can deliver personalized and relevant coaching.

Color-Coding & Nudges: Providing simple visual or haptic prompts (red/yellow/green) can guide users without overwhelming them with technical details.

By embracing the trend of RQ rather than fixating on absolute values alone, this wearable can deliver actionable insights—helping users optimize nutrition, manage exercise intensity, and maintain healthy metabolic states.

Supporting Data and Accuracy. Using de-identified patient data, our device regression model achieves: Classification accuracy of approximately 78% when categorizing RQ into three bins: <0.7, 0.7-1.0, and >1.0. Residual Standard Error of approximately 0.093 for continuous RQ predictions-meaning on average, predicted RQ differs from measured RQ by +0.093.

Features. Direct RQ Measurement. No other available wearable directly measures RQ from transcutaneous CO₂/O₂. Existing market devices rely on proxies like heart rate and accelerometer data.

Algorithmic Integration. The final product integrates multiple signals (PaCO₂, PaO₂, HR, RR, temperature, etc.) into one cohesive regression model that yields an accurate RQ. The device's classification accuracy (78% for RQ bins) is a major leap over standard fitness watch “calorie count” estimates.

Flexible for Multiple Applications. Equally valuable to fitness enthusiasts, clinicians managing metabolic therapies, and sports scientists studying endurance and performance.

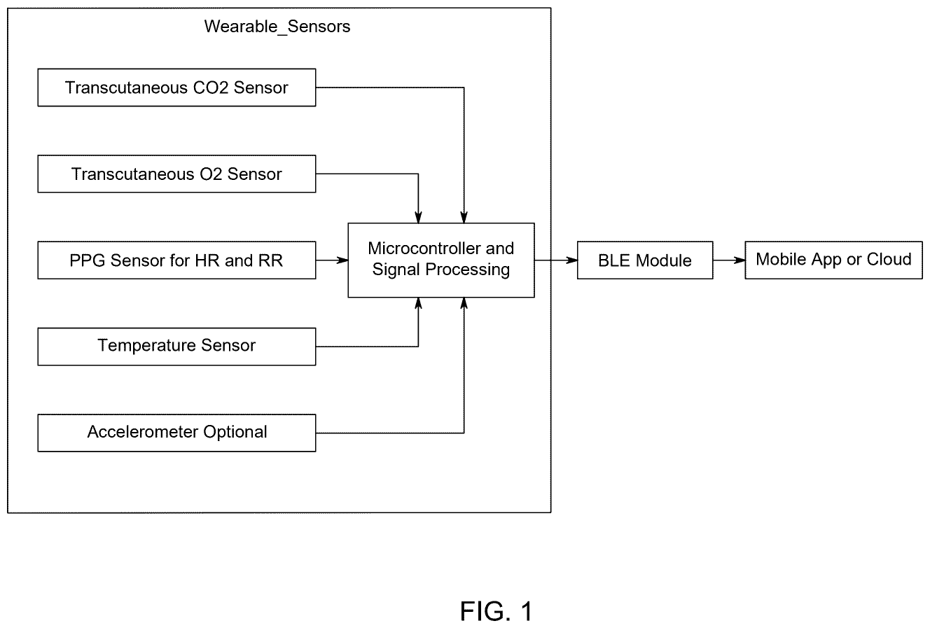

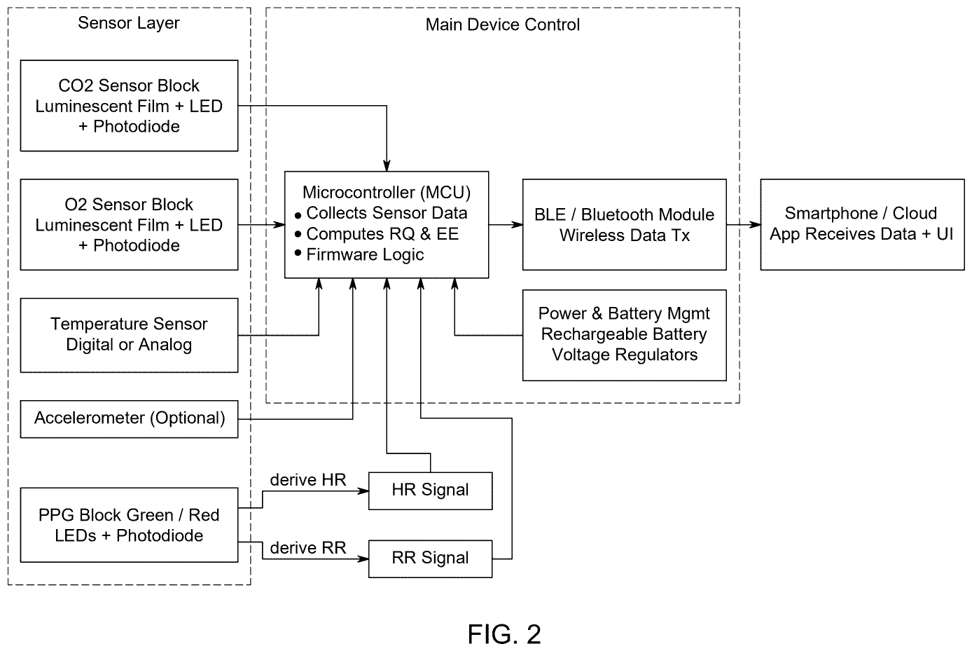

System Diagram: Illustrating the sensor layout (CO₂, O₂ dyes, PPG LED, photodiodes, microcontroller). See FIGS. 1 and 2.

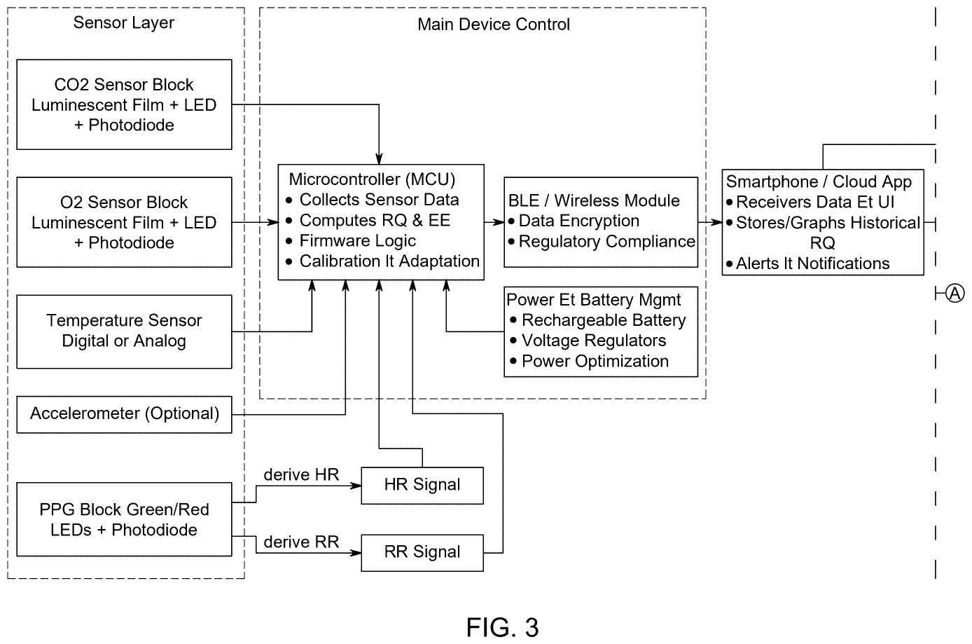

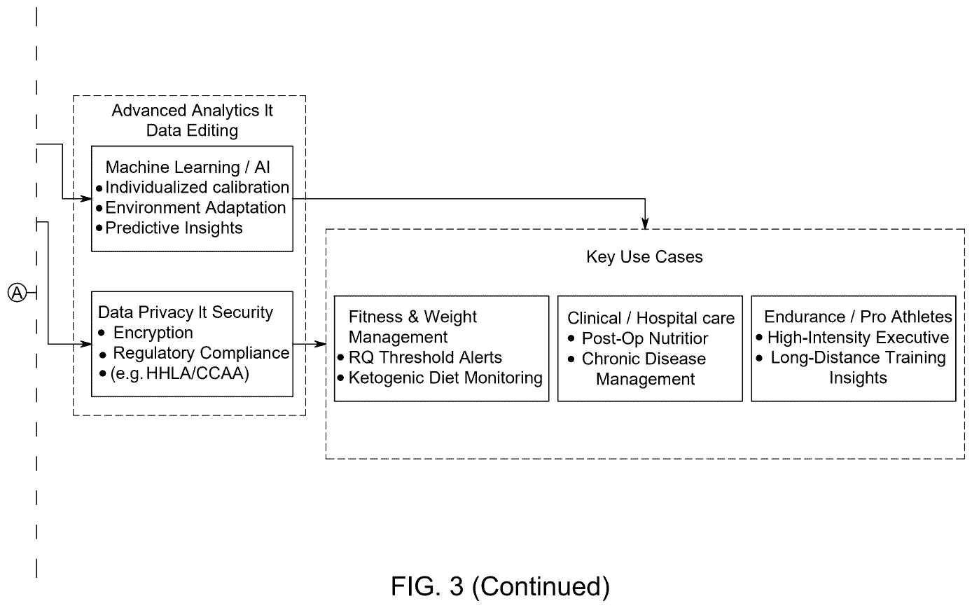

Prototype Rendering: mock-up of the watch/patch. See FIG. 3.

Direct Respiratory Quotient (RQ) Measurement. Most current wearables rely on indirect proxies (heart rate, steps, accelerometer data) and generic formulas to estimate energy expenditure. This device incorporates transcutaneous CO₂/O₂ sensing to directly calculate the user's RQ, which yields a far more accurate assessment of real-time metabolic activity.

Transcutaneous CO₂ (PaCO₂) and O₂ (PaO₂) Sensing. The invention uses luminescent sensor films for CO₂ and O₂, allowing partial pressures to be measured noninvasively through the skin. This sensor-based approach is novel in the consumer/fitness market and represents an advance beyond the PPG- and accelerometer-only methods used by conventional wearables.

Integrated Approach for Multiple Physiological Parameters. While existing trackers might collect heart rate and steps, this invention uniquely combines heart rate, respiratory rate, temperature, and transcutaneous gas measurements into a unified algorithm. This enables the device to compute not only RQ but also more precise energy expenditure (resting, activity-based) and other metabolic parameters in real time.

Versatility and Accuracy. The estimated 78% classification accuracy in three RQ bins (<0.7, 0.7-1.0, >1.0) is significantly higher than ordinary wearables' calorie estimates.

By measuring actual CO₂ and O₂ at the skin level, the invention can adapt to different nutritional states (e.g., ketosis, fasting, high-intensity exercise) and provide more reliable feedback.

Form Factor Variations. Wristwatch-Style. Traditional watch casing with a band (silicone, leather, metal, or polymer). Sensors (CO₂/O₂ films, PPG) on the underside for skin contact. May include a digital screen for displaying RQ, HR, CO₂, O₂, etc.

Adhesive Patch. Thin, flexible patch placed on the upper arm, torso, or even the lower back. Could connect to a small wireless module or a smartphone for real-time readings. Advantageous for continuous monitoring in medical or athletic settings, where wearing a watch might be uncomfortable.

Ring, Clip, or Other Wearable Shapes. A ring that houses transcutaneous sensors, though limited surface area may constrain certain sensor technologies. Finger or earlobe clip attachments: More specialized but potentially convenient for short-term or clinical use.

Sensor & Component Variations. Luminescent Sensor Films. CO₂: HPTS-based or other pH-sensitive dyes (custom chemical formulations possible). O₂: Ruthenium or platinum-based dyes, or alternative phosphorescent materials. Thickness, substrate type (silicone, polymer, glass), and membrane permeability can vary.

PPG Sensor Configurations. Single LED wavelength (e.g., green) for heart rate. Dual-wavelength (green+infrared) or even triple-wavelength for improved respiratory rate extraction or SpO₂ measurement. Placement can shift (top of the wrist vs. underside vs. in a patch).

Additional Sensors. Temperature: Digital thermistor or IR sensor for skin temperature. Accelerometer: To reduce motion artifacts and refine respiratory rate or activity tracking. Gyroscope/Compass: May be included for advanced motion-based features.

Electronics & Microcontroller. Different families of MCUs (ARM Cortex-M, Nordic nRF series, etc.) for power/processing needs. Various wireless modules: Bluetooth Low Energy (BLE), Wi-Fi, or even cellular, depending on use case.

Materials & Construction. Casings and Bands. Metals: Stainless steel, aluminum, titanium (premium look, durable). Plastics/Polymers: ABS, polycarbonate, or TPU for lighter-weight, lower-cost options. Elastomers: Medical-grade silicone or polyurethane for comfortable skin contact.

Gas-Permeable Membrane. PTFE (Teflon) membranes, or specialized polymer layers that allow CO₂/O₂ diffusion but protect the sensor from sweat and moisture. Hydrophobic vs. hydrophilic coatings, depending on the environment (fitness vs. clinical).

Assembly Methods: Injection molding (mass production) vs. 3D-printed prototypes (rapid iteration). Rigid vs. flexible PCBs to accommodate bendable patch designs.

Sealing/encapsulation strategies for water resistance or sterilization (e.g., IP67 rating for consumer devices).

Firmware & Software Variations. Calibration Algorithms.

Built-in calibration once per day (or continuous calibration) based on reference conditions or user input.

Machine-learning models that adapt to individual user physiology over time.

### Data Processing Approaches.

Linear/Polynomial Regression for a straightforward relationship between partial pressures and RQ.

Neural Networks/Advanced Machine Learning to handle more complex variables and interactions (user age, temperature, heart rate variability, etc.).

### User Interface Options.

On-Device Display: Minimal readouts (RQ, HR, etc.) or more detailed user interface.

Companion App: Could display real-time graphs, store historical data, and provide alerts (e.g., “You are in ketosis,” or “High carbohydrate metabolism”).

Cloud Integration: Data sent to a secure server for advanced analytics, sharing with trainers, or integration with medical EHR systems.

Power Management. Different duty-cycling strategies for the sensors and LEDs to optimize battery life. On-demand measurement vs. continuous streaming, depending on user preference or clinical need.

Use-Case and Target Market Variations. Fitness Consumer Market. Sleek, stylish design, possibly with colorful screens. Focus on workout stats and calorie burn accuracy.

Clinical/Medical Market. More robust calibration, regulatory compliance (FDA/CE). May use patches instead of watches for bedridden or post-op patients, real-time monitoring in a hospital or home-care setting.

Professional Athlete/Research Market. Possibly more specialized firmware for high-intensity training. Integration with lab-grade metabolic carts for calibration, or advanced analytics. Weight Management/Ketogenic Diet. Emphasize RQ ranges that indicate fat vs. carbohydrate metabolism (0.6-1.0).

User alerts: “Fat-burning zone,” or “Carb-based metabolism.”

Manufacturing & Scaling Approaches. Low Volume/Pilot Runs. 3D-printed enclosures, off-the-shelf components, small-batch assembly. Ideal for early testing, proof-of-concept, or pilot clinical studies.

High Volume/Mass Production. Customized injection molds, fully automated PCB assembly lines, dedicated sensor film suppliers. Potential for cost reduction and brand partnerships.

Customization/White-Labeling. Could allow third parties to integrate the RQ sensor module into their own watch or patch designs.

The sensor technology has broader use in other health devices. In one embodiment: a full replacement strap for a host smartwatch. The strap includes an underside module positioned so that when the watch is worn normally, the module presses gently against the skin (typically the volar wrist). The module maintains a stable contact area to support gas diffusion through a membrane and to reduce motion artifacts.

Key mechanical requirements: Stable skin contact with controlled pressure (enough to maintain a seal, not enough to cause discomfort). Optional compliant/floating or swivel-mounted sensing head (gimbal/hinge) to maintain consistent normal force and alignment during wrist flexion and motion. Light-blocking and environmental sealing around the sensing window (reduces ambient light and sweat contamination). Replaceable consumable interface (membrane and/or film cartridge) for hygiene and drift management. Compatibility with multiple host-watch lug/connector geometries (via interchangeable adapters or multiple SKUs). Skin-friendly materials and geometries (avoid pressure points; manage sweat; allow airflow). Add-on strap module (a ‘sidecar’ or clip-on module) that mounts adjacent to a host watch without replacing the entire strap. Standalone watch that integrates the sensing module within the watch body. Other wear locations: armband, patch, ring, clip-on, chest strap, or torso patch.

FIG. 1 shows a General Schematic Diagram of RQ Fitness Wearable.

FIG. 2 shows a Detailed Schematic Diagram of RQ Fitness Wearable.

FIG. 3 shows an RQ Fitness Wearable Technical Architecture and Potential Use Cases.

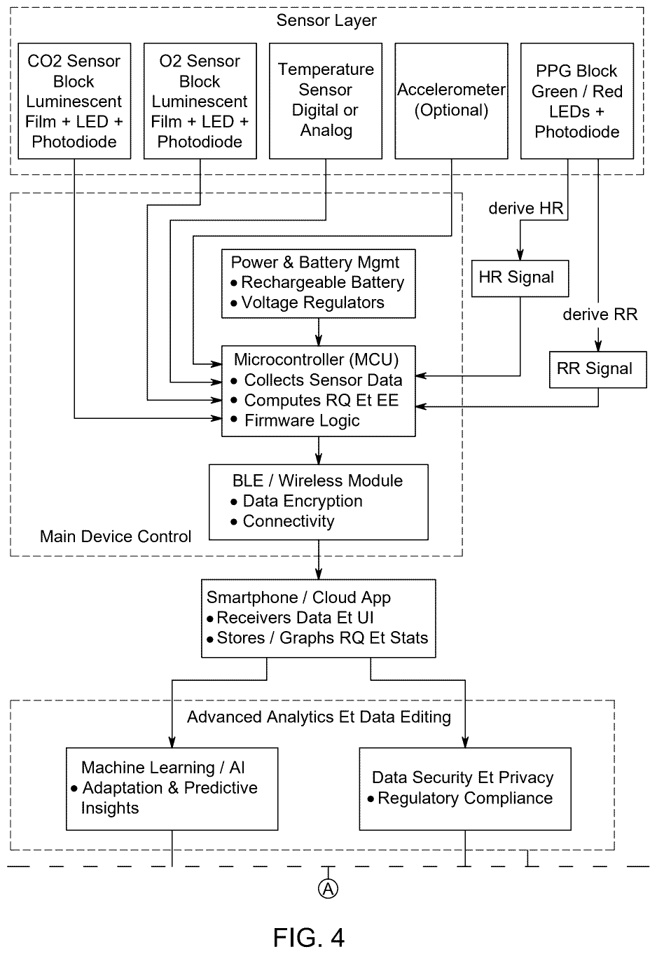

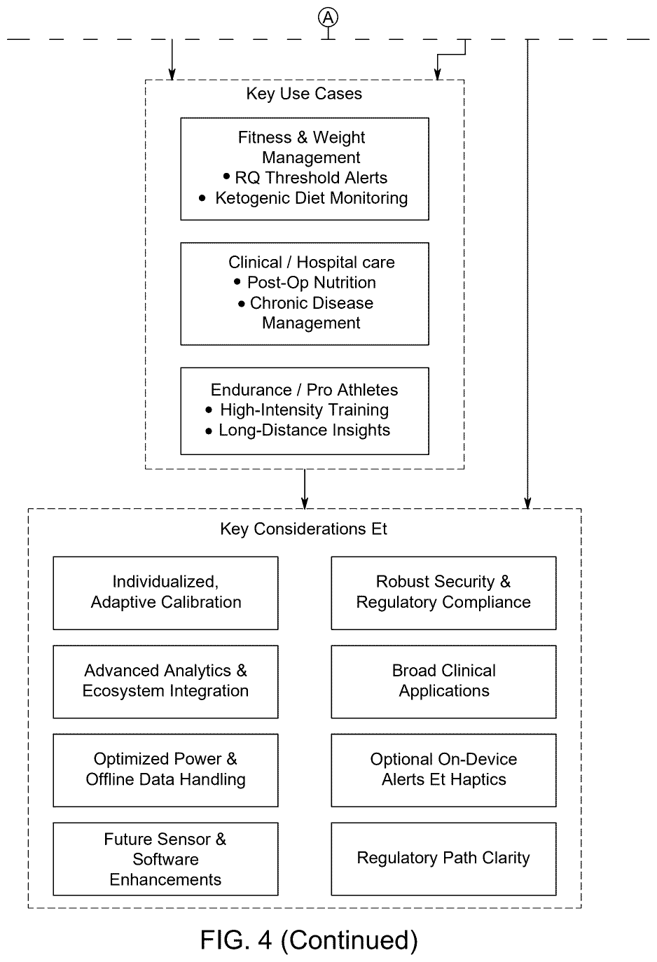

FIG. 4 shows a Comprehensive RQ Wearable Architecture: Sensors, Use Cases, and Key Considerations.

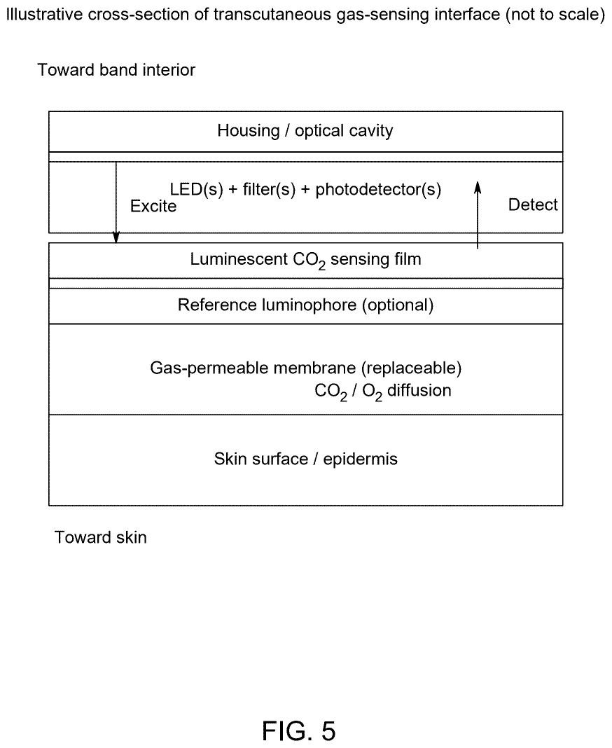

FIG. 5 shows an exemplary cross sectional diagram of a transcutaneous gas-sensing interface of the disclosure.

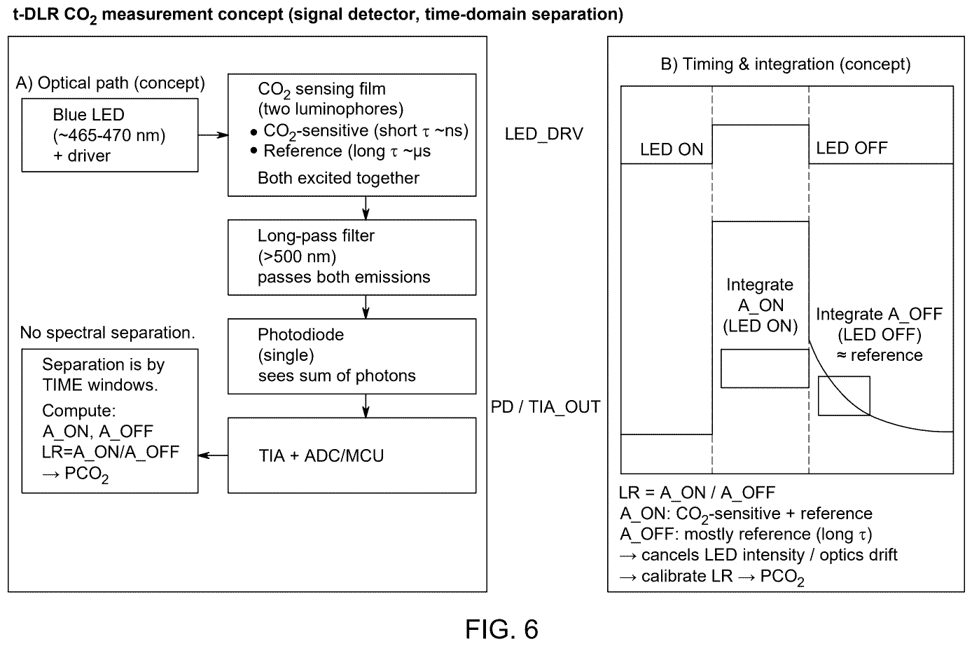

FIG. 6 shows an embodiment of t-DLR CO₂ measurement.

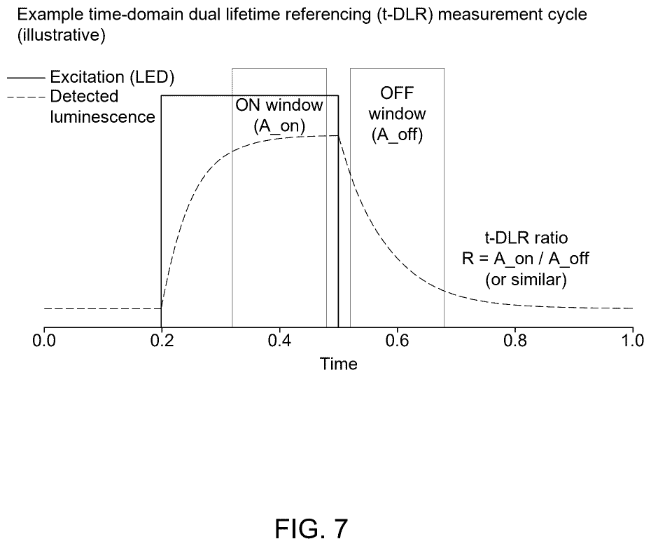

FIG. 7 shows an Example time-domain dual lifetime referencing (t-DLR) measurement cycle.

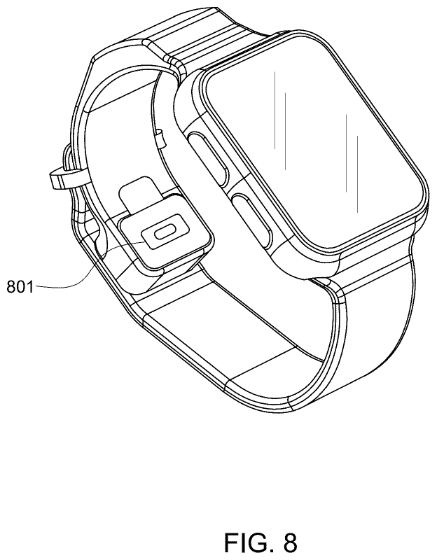

FIG. 8 shows an Example of a smartwatch embodiment of the disclosure showing sensor 801.

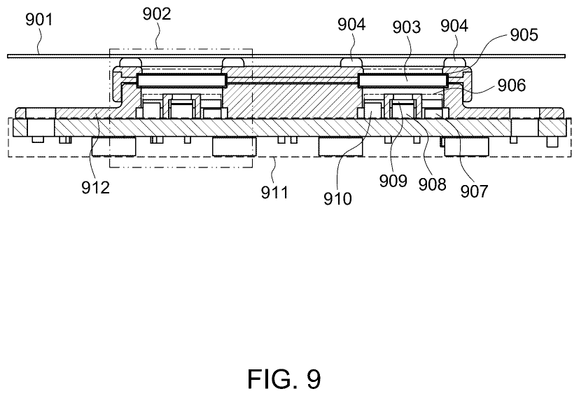

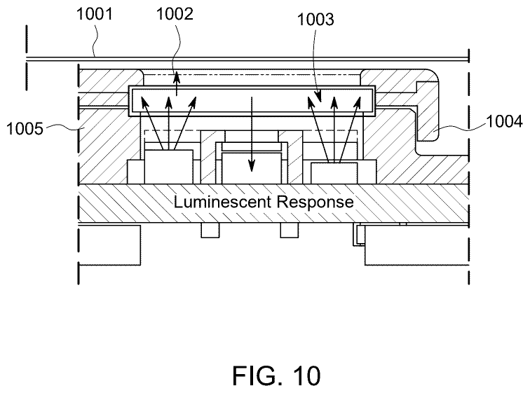

FIGS. 9 and 10 show a sensing interface cross-section—membrane+sensing film(s)+optics; illustrates gas diffusion path, optical excitation/emission paths, and an example t-DLR-compatible LED/filter/photodiode layout (with optional O₂ channel).

FIG. 9 shows skin surface 901; optional O2 sending module 902; CO2 sensing element 903; optional seal/gasket/hydrogel ring 904; gas permeable membrane 905; optical cavity 906; excitation LED 907; photodiode (single detector) 908; long-pass filter (>500 nm); excitation LED (405 nm); TIA/AFE+ADC/MCU (electronics region); and sensing module enclosure/housing 912.

FIG. 10 shows skin surface 1001; CO2 diffusion (skin sensor); CO2 exchange/back diffusion 1003; excitation light (e.g., 470 nm) 1004; and excitation light (e.g., 405 nm) 1005.

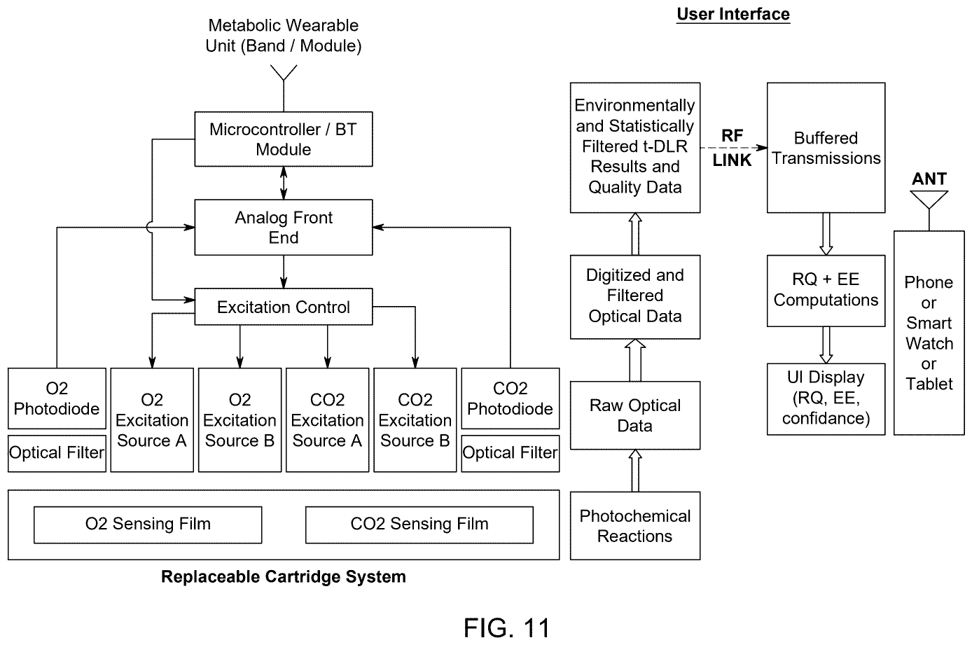

FIG. 11 shows an example processing pipeline and electronics blocks (AFE, t-DLR, RO/EE computation, UI).

Skin-contact sensing interface (membrane+films+optics). The sensing module includes a skin-contact sensing interface designed to measure gases that diffuse through the skin—primarily CO₂ and optionally O₂—without puncturing the skin. In typical use, skin-diffused gases pass through a gas-permeable membrane into a sensing element (one or more films or layers) that converts local CO₂ (and optionally O₂) conditions into an optical signal (fluorescence and/or phosphorescence). The module's optics and electronics measure that optical signal and produce one or more gas-related features that are later used to compute RQ and EE.

Functional layer stack. A typical interface can be described as the following functional layers (from skin outward). The ordering, thickness, exact materials, and integration approach can vary: Skin contact/seal region. A compliant ring, gasket, diaphragm, hydrogel, or other interface can be used to improve contact stability, reduce sweat ingress, and reduce motion artifact.

Gas-permeable membrane. A membrane permits diffusion of CO₂ (and optionally O₂) while acting as a barrier to liquid water and sweat. The membrane may be hydrophobic and/or porous, and may be part of a replaceable consumable.

CO₂ sensing element (required for CO₂ channel). The CO₂ sensing element may be implemented as one or more films, layers, or printed regions that respond to CO₂ (often via pH chemistry). Important for t-DLR embodiments: the CO₂ sensing element may include two luminophores in a single sensing region: A CO₂-sensitive luminophore (short lifetime, e.g., ns-scale), and a CO₂-insensitive reference luminophore (long lifetime, e.g., μs-scale), so that a single optical detector can measure the combined emission and the components can be separated using time windows.

O₂ sensing element. An optional O₂ sensing film may be included as a separate layer/region or integrated into a multi-layer stack. Depending on the luminophore, the O₂ film can be read by intensity, phase, lifetime, or other methods.

Optical window/cavity/mechanical support. A transparent window, spacer, or optical cavity may separate the sensing films from the LED/photodiode components while maintaining optical coupling and mechanical protection.

Optics and detector. The sensing module includes one or more excitation light sources (LEDs) and at least one photodetector (often a photodiode).

In certain embodiments (including t-DLR), a long-pass filter (e.g., >500 nm cutoff) blocks the excitation wavelength (blue) and passes the combined emissions. This supports a single-detector approach in which separation is performed by timing, not wavelength.

Illustrative cross-section. Conceptual layer stack for a transcutaneous CO₂ (and optional O₂) sensing interface on the underside of a wearable band. The membrane, sensing element(s), and optical components may be arranged in different orders, thicknesses, and materials; the disclosure should cover alternatives including multi-layer films, printed films, and replaceable consumables.

Recommended callouts to include. Skin surface. Seal/gasket/hydrogel ring. Gas-permeable membrane (water/sweat barrier). CO₂ sensing element (indicator+optional reference luminophore). Optional O₂ sensing element. Optical window/spacer/cavity. Blue excitation LED (~470 nm). Long-pass filter (>500 nm). Photodiode (single detector). TIA/AFE+ADC/MCU (electronics region).

Membranes (gas-permeable barriers). PTFE, ePTFE, silicone, polyurethane, fluoropolymers, porous laminates, multilayer composites; Hydrophobic membranes and/or textured/porous structures to reduce sweat ingress. Optional anti-fouling or skin-compatible coatings; Thickness and porosity are design variables.

CO₂-sensitive chemistries. pH-sensitive dye systems (e.g., HPTS-based); other optical CO₂ indicator systems (including but not limited to pH-mediated, ion-pair, or alternative solid-state CO₂ indicators); polymer matrices, buffers, immobilization strategies, and stabilizers; printed or coated films; patterned regions; multilayer constructs;

Reference luminophore. CO₂-insensitive reference dyes (e.g., ruthenium-based or other stable luminophores) co-located in the same film as the CO₂ indicator, or placed as a separate layer/region that is optically coupled to the same detector path. Selected to have a substantially longer effective lifetime than the CO₂-sensitive luminophore in the readout configuration.

O₂-sensitive chemistries. ruthenium, platinum, or other oxygen-quenchable luminophores. intensity-, phase-, or lifetime-based readout; separate excitation wavelength or time-multiplexed excitation.

Optics/electronics variations. single photodiode+long-pass filter (time-domain separation); multiple photodiodes and/or additional filters (spectral separation); ambient light rejection via baseline sampling and subtraction; mechanical baffles, reflectors, optical guides, and shielding to reduce ambient coupling.

Replaceable consumables. replaceable cartridges/caps/patches that integrate membrane+sensing element(s); single-use or multi-day consumables; alignment features, peel liners, blister packaging, applicator tools; disposable membrane-only with reusable optics, or fully disposable membrane+film stack.

Link to t-DLR timing. In embodiments using t-DLR, the interface is designed so that the CO₂-sensitive luminophore and the reference luminophore are both excited by the same LED pulse and detected through the same optical channel, allowing time-windowed processing to generate a referenced CO₂ metric that is less sensitive to LED intensity drift, optical coupling changes, and detector gain changes.

FIG. 5 shows an Illustrative cross-section. Example layer stack. Actual order, thickness, and materials may vary.

Materials and variations. Gas-permeable membrane materials: PTFE, silicone, polyurethane, fluoropolymers, or multilayer laminates; optionally hydrophobic to block sweat/water. CO2-sensitive chemistry: pH-sensitive dye systems (e.g., HPTS-based), other optical CO2 indicators, or solid-state chemistries; various polymer matrices and buffers. O2-sensitive chemistry: ruthenium, platinum, or other oxygen-quenchable luminophores; intensity-, phase-, or lifetime-based readout. Reference luminophore: CO2-insensitive dye used for referencing/normalization. Replaceable cartridges/caps/patches that integrate membrane+films as a single consumable.

Optical engine/opto-mechanical carrier for LED+filter+detector+removable sensing element. The sensing module may include an opto-mechanical carrier (“optical engine”) configured to mechanically support, align, and protect an optical sensing path comprising at least: (i) an excitation light source (e.g., LED), (ii) a photodetector (e.g., photodiode), (iii) one or more optical filters configured to attenuate excitation light while passing luminescent emission (e.g., a long-pass filter with a cutoff near the excitation wavelength), and (iv) a removable sensing element such as a sample disk, cap, or cartridge carrying a gas-permeable membrane and one or more sensing films (e.g., CO₂ indicator film and optional reference luminophore; optional O₂ film).

In certain embodiments, the optical engine defines fixed or controlled optical spacing between the LED/detector and the sensing element, provides ambient-light shielding and excitation-light rejection, and maintains repeatable alignment across module assembly and consumable replacement. In certain embodiments, the optical engine supports a single-detector architecture in which combined emissions from multiple luminophores are measured along a single optical path and processed using time-windowed sampling (e.g., t-DLR).

Carrier features include Alignment and keying: pins, bosses, datum surfaces, kinematic mounts, keyed geometry, and/or asymmetric features that enforce repeatable orientation of a sample disk/cartridge.

Retention: snap-fit, screw, clamp, press-fit, adhesive, ultrasonic welding, laser welding, or over molding that retains optics and/or the sensing element. Sealing: gaskets, O-rings, diaphragms, hydrogel interfaces, and/or labyrinth seals around the sensing window to reduce sweat ingress and stabilize contact. Stray-light control: optical baffles, light traps, apertures, opaque partitions, blackened/absorptive surfaces, and/or reflective cavities to reduce excitation leakage to the photodetector and reduce ambient coupling.

Receptacle for consumables: a pocket, seat, shoulder, or ledge that positions the sample disk/cartridge with repeatable compression and standoff, optionally including compliance features (springs/foam/elastomer) to maintain optical coupling under strap pressure and motion.

Filter placement options: the emission filter may be a discrete component retained in the optical engine, bonded/laminated to the photodiode, integrated into a window, or implemented as a coated optical element, with equivalent function.

Manufacturing variations: the carrier may be injection-molded polymer, machined metal, molded/printed polymer, or a multi-material assembly (e.g., rigid carrier with elastomer seals).

The optical engine is coupled to an analog front end (AFE) configured to drive the excitation LED with pulses and synchronously sample the photodetector signal to compute ON/OFF window features (e.g., A_ON and A_OFF) and referenced metrics (e.g., A_ON/A_OFF).

Removable sensing element/consumable packaging variations (disk, cartridge, or membrane-sealed pocket)

In various embodiments, the CO₂ sensing element (and optional reference luminophore) is provided as a replaceable consumable that interfaces with the optical engine. The consumable can be replaced for hygiene, to mitigate long-term drift/aging, and to accommodate limited chemical lifetime of the sensing chemistry. The disclosure should cover multiple non-limiting packaging forms, including solid disks/pads and membrane-sealed pockets containing particulate or microsphere-based reagents.

Non-limiting consumable form factors: Solid sensing disk/pad: a polymer disk or laminated film stack containing (i) a CO₂-sensitive luminophore system (often pH-mediated) and (ii) an optional long-lifetime reference luminophore for t-DLR. Cartridge/cap: a rigid or semi-rigid frame that carries the membrane+sensing element(s) and mates to the band module with keying/alignment features.

Membrane-sealed pocket (“pouch”): a sealed cavity that contains dye-loaded microspheres or other particulate sensing media in a granular/powder form, optionally with buffer/polymer support, enclosed between two membranes/films.

Example membrane-sealed pocket construction. Skin-facing membrane: gas-permeable, sweat/water-resistant material (e.g., PTFE/ePTFE, silicone, polyurethane, fluoropolymer laminates) that contacts skin. Optical-facing film: optically clear and gas-permeable film (or multilayer laminate) that allows excitation/emission light to pass while retaining the particulate sensing media. Perimeter seal: heat seal, ultrasonic weld, laser weld, adhesive bond, over molded gasket, or mechanical clamp ring that forms a sealed pocket to retain the media and define a repeatable diffusion path.

Pocket geometry: flat, domed, ring (“donut”) geometry, or multi-compartment geometry; may be supported by a carrier ring to maintain flatness and repeatable optical alignment.

Process variables: film stretch/strain during sealing (used to form a pocket volume), seal width, pocket thickness, and media loading (mass/volume) as controlled design/manufacturing variables. In some embodiments, the consumable includes an identifier (e.g., QR/ID, RFID/NFC tag, or a coded mechanical key) that allows the band/watch/app to select the appropriate calibration mapping for that consumable type/lot.

Response time and diffusion considerations (double-diffusion) and optional acceleration features. Transcutaneous gas sensing in a wearable can be limited by gas transport across multiple boundaries and compartments, including blood/capillary to tissue, tissue to epidermis, across the stratum corneum, across the skin-interface boundary, through any membrane or barrier layer, and into the sensing film. These serial transport resistances can slow step response, smooth rapid changes, and create site-to-site variability. Accordingly, the invention includes design features that improve response time, reduce stagnant boundary layers, and improve contact stability.

Non-limiting response-time improvement features: Minimize diffusion distance: use thin membranes/films, thin sensing layers, and reduced pocket thickness/volume. Increase gas permeability: select high-permeability membranes or multilayer laminates optimized for CO₂/O₂ diffusion while resisting liquid ingress. Improve and maintain contact: compliant seals, controlled strap pressure, and/or a floating/swivel-mounted sensing head that maintains consistent normal force as the wrist flexes. Local thermal control: a micro-heater or thermoelectric element to gently warm the measurement site to increase perfusion and gas flux (with safety limits and duty-cycling). Geometry/channel control: microstructured membranes, diffusion channels, or boundary-layer management features that reduce stagnant volumes and improve equilibration.

Algorithmic handling: confidence gating and a defined warm-up/stabilization period before using readings for RQ/EE outputs. Optical readout using time-domain dual lifetime referencing (t-DLR)

Time-domain dual lifetime referencing (t-DLR) is an optical readout technique that uses two luminophores with very different lifetimes in the same sensing element: CO₂-sensitive luminophore (short lifetime; typically, nanoseconds scale), whose emission amplitude (or effective brightness) changes with CO₂ (often via pH chemistry).

### Example: HPTS-Based Indicator Chemistry

Reference luminophore (CO₂-insensitive; long lifetime; typically, microseconds scale), used to normalize out common drift.

### Example: A Ruthenium Complex Reference Dye

Both luminophores are excited by the same LED pulse. A single photodiode measures the sum of their emitted photons. Separation of the two components is achieved by time windows, not by wavelength.

t-DLR produces a referenced metric (e.g., LR=A_ON/A_OFF) that is designed to reduce sensitivity to: LED brightness changes over time; optical coupling changes (skin contact, strap pressure, motion); detector gain drift/fouling/partial occlusion; some (not all) aging and alignment effects.

Optical stack (typical implementation for a wrist band). A typical t-DLR optical stack for CO₂ sensing in the band includes Excitation LED (example wavelength: ~470 nm). The LED pulse excites both luminophores simultaneously. CO₂ sensing film containing two luminophores. CO₂-sensitive luminophore (short lifetime, τ_sens~ns). Reference luminophore (long lifetime, τ_ref~μs). The CO₂-sensitive luminophore changes with CO₂/pH; the reference does not. Long-pass emission filter (example cutoff: >500 nm). Blocks blue excitation light. Passes both emission bands (e.g., ~520 nm indicator emission and ~600-620 nm reference emission). No spectral separation is required for t-DLR; the photodiode can see the combined emission. Single photodiode Measures the sum of the emitted photons: I(t)=I_sens(t)+I_ref(t).

Analog front end (TIA)+ADC+processor. Converts photodiode current into a sampled waveform (or sampled windows) that can be integrated. Timing and window definitions (what gets measured)

t-DLR is implemented with a repeating cycle that includes at least: A) LED ON window→A_ON. The LED is turned ON for a controlled time. During this ON period, both luminophores emit simultaneously. The photodiode measures the combined emission, and the system integrates (or averages) the signal over a defined ON window: A_ON (LED ON window). A_ON is the integrated (or averaged) photodiode signal during the LED-ON period. During this time, both the CO₂-sensitive luminophore (short lifetime) and the reference luminophore (long lifetime) emit simultaneously, so the detector measures their combined emission. Because the CO₂-sensitive component changes with CO₂ (often via pH chemistry), A_ON varies with CO₂. B) LED OFF window→A_OFF. The LED is turned OFF. The short-lived luminophore decays essentially immediately (ns-scale), while the long-lived reference continues emitting (μs-scale).

After a short delay to allow electronics to settle (and to ensure short-lived emission is negligible), the system integrates over a defined OFF window: A_OFF (LED OFF window). A_OFF is the integrated (or averaged) photodiode signal during a post-excitation LED-OFF window. After the LED turns off, the short-lifetime emission decays essentially immediately, while the long-lifetime reference continues to emit. By starting A_OFF after a short post-OFF delay, the system avoids LED switching/electronics transients, so the OFF window is dominated by reference emission. A_OFF therefore serves as a CO₂-insensitive normalization signal that tracks common-mode variations such as LED intensity drift, optical coupling changes, and detector gain changes.

Practical note (why we wait briefly after LED-OFF): When the LED turns off, the analog chain (LED driver→photodiode→TIA/AFE→ADC) can exhibit a short switching transient and may take a brief time to settle. Although the CO₂-sensitive dye decays on a nanosecond timescale, the measured waveform can be limited by analog bandwidth and sampling. Accordingly, the firmware may insert a short guard/settle delay after LED-OFF before integrating A_OFF, so that A_OFF captures the long-lifetime reference afterglow rather than electronics artifacts.

The referenced t-DLR metric (why drift cancels). t-DLR deliberately avoids needing two separate photodiodes and two separate emission filters. Instead, the photodiode measures the *sum* of photons from (i) the CO₂-sensitive luminophore (short lifetime) and (ii) the CO₂-insensitive reference luminophore (long lifetime). Time windows—rather than wavelength separation—are used to separate those contributions.

Define a baseline-corrected photodiode waveform I(t) for each LED pulse cycle. During the LED-ON window, the measured signal contains both contributions: I(t)≈I_sens(t)+I_ref(t).

$$A\_ON = \int l ( t ) dt over the LED - ON window \approx \int ( l\_sens ( t ) + l\_ref ( t ) ) dt$$

A_OFF=∫I(t) dt over the LED-OFF window (after a short guard/settle delay)≈∫I_ref(t) dt

Interpretation: • A_ON depends on CO₂ because the CO₂-sensitive luminophore's emission changes with CO₂ (often via pH chemistry). • A_OFF is designed to be reference-dominated (CO₂-insensitive) and therefore tracks common-mode factors such as LED output, optical coupling, filter transmission, photodiode responsivity, and analog gain.

A common referenced metric is the luminescence ratio: LR=A_ON/A_OFF.

Why the ratio cancels drift (intuitive): if a multiplicative factor G represents changes in LED brightness, optics, detector gain, and/or fouling, then A_ON≈G·(A_sens+A_ref) and A_OFF≈G·A_ref, so LR≈(A_sens+A_ref)/A_ref=1+(A_sens/A_ref). Thus, (LR−1) behaves like the indicator strength relative to the reference and is far less sensitive to common-mode drift than raw intensity.

The device maps LR (or LR−1, or another referenced parameter) to a transcutaneous CO₂ metric using a calibration curve and compensation models. Ambient/baseline subtraction (recommended for wearables). Wearable optical sensors must tolerate changing ambient light and DC offsets. A practical approach is to measure a baseline with the LED OFF and subtract it from subsequent samples before integrating windows.

One example per pulse cycle: Baseline window (LED OFF): measure I_base (ambient+offsets); Signal sampling: measure I_meas(t) during LED-ON and LED-OFF Baseline-corrected waveform: I(t)=I_meas(t)−I_base Compute A_ON and A_OFF from I(t).

Baseline subtraction improves robustness to ambient leakage, strap-motion light coupling, and slow changes in sensor offsets.

Implementation (what the band electronics do). Typical processing steps (non-limiting): Pulse the excitation LED at a defined repetition rate and duty cycle (typically in the blue range for HPTS-class indicators, though alternatives are covered). Acquire photodiode output with time synchronization to the LED pulse using a TIA/AFE and ADC (see Section 5.3.8 for AFE considerations).

Measure an ambient/baseline level during an LED-OFF window and subtract it to form a baseline-corrected waveform I(t). Integrate (or average) I(t) over a defined LED-ON window to obtain A_ON. After LED-OFF, wait a short guard/settle delay, then integrate (or average) I(t) over a defined LED-OFF window to obtain A_OFF (reference-dominated). Compute a referenced metric, e.g., LR=A_ON/A_OFF. Variants include differences (A_ON−k·A_OFF), weighted ratios, multi-window features (early-off vs late-off), or exponential/lifetime fits. Convert the referenced metric to a transcutaneous CO₂ estimate via calibration and compensation (temperature compensation, aging/drift compensation, and contact-quality compensation). Output the CO₂ metric and associated confidence/quality indicators to the RQ and EE estimation pipeline.

Relationship to O₂ sensing. If an O₂ sensing film is included, it may be read using any suitable optical method, including separate excitation wavelength(s) (e.g., a second LED), time multiplexing (alternate CO₂ and O₂ measurement cycles), and/or lifetime/phase techniques appropriate for the chosen O₂ luminophore.

CO₂ (t-DLR) and O₂ measurements may share mechanical packaging (membrane, optical window, carrier) while using separate excitation timing and/or processing to reduce crosstalk.

t-DLR is described primarily for the CO₂ channel, but the architecture can support multiple channels using scheduled excitation/sampling. In some embodiments, RQ and EE are computed without any direct O₂ measurement, using CO₂-derived features and other physiological context signals alone.

Example t-DLR cycle using a single excitation LED and a single photodiode. A_ON is integrated during the excitation pulse and includes both the CO₂-sensitive and reference emissions. A_OFF is integrated after excitation is turned off and is dominated by the long-lifetime reference. The ratio LR=A_ON/A_OFF provides a referenced metric that reduces sensitivity to LED intensity drift and optical/electronic gain variation. Exact timing, delays, window durations, and mapping are implementation choices.

### Illustrative Timing Diagram.

Implementation notes (what the band electronics do): Drive one or more excitation LEDs at defined pulse frequency and duty cycle. Sample photodetector output (or AFE output) at fixed offsets to form ON-window and OFF-window integrals/averages. Compute a referenced metric (e.g., ratio, difference, lifetime fit, or model-fit parameters) correlated with CO₂ partial pressure in the sensing film environment. Apply calibration/compensation: temperature compensation, sensor aging compensation, and optional per-user calibration. Optionally multiplex wavelengths (CO2 film vs O2 film vs reference) via time-multiplexing and/or optical filtering. Analog front end (AFE) for time-synchronized t-DLR sampling (non-limiting)

The sensing module may include an analog front end (AFE) configured to (i) drive one or more excitation LEDs with controlled pulse width, amplitude, and repetition rate and (ii) acquire a photodetector signal in time synchronization with the excitation pulses. In t-DLR embodiments, the AFE supports sampling during both an LED-ON period and an LED-OFF decay period, thereby enabling computation of windowed integrals or averages (e.g., A_ON and A_OFF), optional baseline/ambient subtraction, and a referenced metric such as LR=A_ON/A_OFF.

The AFE may include one or more of: a programmable LED current driver (or driver+external switch transistor); a transimpedance amplifier (TIA) for the photodiode; programmable gain and offset adjustment; an ADC (internal or external); timing/synchronization circuitry (MCU timer, FPGA, or AFE timing engine); low-power modes (e.g., TIA disable/shutdown between pulses)

Calibration, compensation, and data quality. Wearable transcutaneous sensing is exposed to real-world variability: strap pressure changes, motion, ambient light leakage, sweat, skin differences, temperature changes, and sensor aging.

This section describes how the system maintains usable accuracy and how it detects/flags low-quality data.

What gets calibrated (CO₂ channel, optional O₂ channel, and optics) A) CO₂ channel calibration (core).

Map a referenced optical metric (e.g., LR=A_ON/A_OFF or a derived value such as LR−1) to a CO₂ metric (e.g., transcutaneous CO₂/ptcCO₂/CO₂ partial pressure at the sensing interface).

Factory procedure examples: Expose the sensing interface (or a representative film/membrane assembly) to known CO₂ partial pressures across a relevant range and at controlled temperatures. Build and store a calibration curve or lookup table: LR→CO₂ (optionally parameterized by temperature). Store per-module or per-cartridge calibration identifiers (e.g., a cartridge QR code or EEPROM ID) to select the appropriate calibration table.

B) Optical/electronics calibration LED output and photodiode/TIA chain may vary by unit. Factory calibration may include LED drive verification (pulse amplitude, timing, repetition rate). Photodiode dark current and baseline offsets. Gain/offset factors for the analog front end (TIA) and ADC. C) Optional O₂ channel calibration.

If an O₂ sensing film is included, calibrate it using known O₂ environments (gas or dissolved oxygen equivalents), and store the mapping for the chosen readout method (intensity, lifetime, phase, etc.).

In-use compensation (what happens on the wrist). A) Temperature compensation. Include a temperature sensor near the sensing window. Use temperature to: adjust the CO₂ mapping (LR→CO₂), compensate luminescence temperature dependence, stabilize computations across cold/warm environments.

B) Drift/aging compensation using the reference luminophore. A key benefit of t-DLR is that the reference component (A_OFF) tracks common changes such as: LED intensity drift, optical path changes (minor misalignment, fouling, partial occlusion), photodiode/TIA gain changes.

Use this in multiple ways: The ratio LR=A_ON/A_OFF inherently normalizes many common-mode drifts. Track long-term trends in A_OFF or LR baseline to detect sensor aging and prompt replacement of the consumable if needed.

C) Ambient light rejection Use one or more LED-OFF baseline samples (ambient window) and subtract from the LED-ON/decay samples to remove: ambient leakage, DC offsets, slow background changes.

D) Contact-quality and seal-quality detection because the sensor is in a band, contact varies. Detect contact quality using: Signal-based indicators: low A_ON amplitude (weak coupling), abnormal LR values or unstable LR variance, excessive baseline/ambient contribution, unexpected decay shape (e.g., motion or optical leakage). Context sensors (optional but recommended):

IMU activity level (high motion=lower confidence), temperature stability (sudden changes can indicate loss of contact), pressure/force sensor or impedance sensor at the module to detect contact. E) Motion artifact handling.

Use IMU features to gate measurements (only compute “high-confidence” CO₂ in stable windows), apply adaptive filtering/smoothing, lower confidence score when movement is high.

F) Optional user-specific respiratory amplitude calibration (A_rest) for ventilation-derived features.

In embodiments that estimate breathing depth or ventilation (e.g., minute ventilation V_E or alveolar ventilation V_A) from motion/respiration signals, the system may perform a one-time (or periodic) user calibration to establish a baseline breath-amplitude reference. This improves robustness to band fit, wrist anatomy, and sensor coupling, and enables personalized ventilation-derived features.

When to calibrate on first use, after strap fit changes, after consumable replacement, and/or periodically. In embodiments, guide the user to sit quietly and breathe normally for a short interval during low motion and stable signal quality (hand and wrist may be placed over the chest for better accuracy); automatically detect a ‘true rest’ window using IMU and physiologic stability criteria.

What is stored: A_rest, a robust statistic (e.g., median) of a breath-amplitude metric A_now measured during the calibration window (the amplitude metric may be derived from IMU, PPG respiration, optical waveform features, or combinations). How it is used: normalize subsequent breath-amplitude estimates (e.g., A_now/A_rest) to obtain a dimensionless depth proxy that can be mapped to ventilation-related features. Safeguards: suppress or down weight ventilation-derived features when motion/contact quality is low; optionally blend the normalized amplitude back toward 1.0 outside of contexts consistent with elevated ventilation.

Output quality flags (so models don't garbage-in/garbage-out). For each CO₂ feature (and optionally O₂), compute: a quality score (0-1 or 0-100), a confidence tag (high/medium/low), optionally a reason code (motion, ambient light, low contact, sensor warming up, cartridge end-of-life).

Downstream RQ/EE models should: ignore or down weight low-quality intervals, carry forward last reliable values cautiously (with clear U indication), log raw+quality metadata for debugging and improvement. Consumable QC, lot traceability, and calibration strategy (avoid per-disc calibration) For manufacturability and scale, it is advantageous to avoid a bespoke calibration curve for every individual consumable disk/pouch. Instead, the system can rely on a combination of controlled manufacturing processes, lot-level characterization, and quality control checks to ensure consistency.

Non-limiting QC and traceability approaches to include in-process optical QC: illuminate each consumable (or a statistical subset) with the intended excitation and verify that the emission response of the indicator and reference falls within acceptance limits (intensity and/or referenced LR metric).

Seal integrity QC for membrane-sealed pockets: visual inspection, leak testing, or optical signature checks that correlate with seal quality and pocket thickness. Lot/batch identification: encode a lot ID on packaging (QR/barcode), on the cartridge body (laser mark), or electronically (RFID/NFC), enabling selection of the correct lot-level calibration mapping.

Insertion self-test: when a consumable is installed, the band can run a short self-test to verify optical coupling, baseline signal levels, and that LR falls within an expected range before reporting measurements.

Configuration management: store consumable type/lot metadata in the band/watch/app so post-market performance can be monitored and models refined. Calibration can then be applied at the module level (device) and/or at the lot level (consumable), optionally with temperature compensation and reference-based drift correction during use. This approach supports high-volume production while maintaining measurement quality.

Longevity, half-life characterization, and end-of-life handling. Because optical sensing chemistries can age (photobleach, leach, dry out, or otherwise drift), the disclosure should cover both (i) how longevity is characterized and (ii) how the system detects end-of-life in real use.

Non-limiting longevity characterization tests: Baseline fade test: expose a consumable to an inert environment (e.g., nitrogen) and monitor referenced signal stability under controlled temperature to quantify photobleaching/aging independent of CO₂ changes. Continuous CO₂ exposure test: expose to controlled CO₂ partial pressure and track the referenced metric (e.g., LR) over time to estimate functional half-life and drift rate. Environmental stress tests: humidity/sweat exposure, temperature cycling, and mechanical flexing tests to evaluate membrane integrity and optical stability.

Non-limiting in-field end-of-life strategies: Track cumulative LED on-time and estimate remaining consumable life based on validated aging models. Monitor reference intensity and LR stability; if SNR or LR falls outside thresholds for a sustained period (with good contact), flag the consumable as degraded. Prompt the user to replace the consumable when confidence falls below a threshold or after a predefined wear interval.

Signal processing pipeline (raw→features→RQ/EE). High-level pipeline: Acquire raw optical signals from the gas-sensing module (CO2 and optionally O2). Compute referenced features using t-DLR (and/or lifetime/phase methods). Acquire physiological context signals: HR, RR, SpO₂, skin temperature, motion/activity features, and optionally demographics (age, sex, height, weight, BMI). Optionally derive breathing depth/amplitude features and ventilation proxies (e.g., V_E and/or V_A) from IMU/accelerometer signals and/or respiration signals; optionally normalize using a stored rest baseline A_rest. Optionally derive a CO2 output feature (e.g., a VCO2 proxy) by combining ptcCO2 with ventilation-related features for use by the RQ and/or EE models.

Filter and quality-control signals: contact detection, ambient light rejection, motion artifact rejection, confidence scoring.

Compute RQ and EE using a trained model (regression or ML). Provide outputs and confidence indicators to the host watch/app; log data for trends. RQ estimation model (inputs, not coefficients). RQ is a co-equal primary output. RQ is computed by a multivariable model trained on reference measurements (e.g., indirect calorimetry and/or blood-gas-based measurements). In some embodiments, RQ and EE are computed without any direct O₂ measurement, using CO₂-derived features and other physiological context signals alone.

Example RQ model inputs (from current modeling work): Transcutaneous CO₂ estimate (ptcCO₂) and/or a CO₂ metric correlated with transcutaneous and/or arterial CO₂. In model development and validation, arterial CO₂ (PaCO₂) may be used as a reference measurement and/or training label. Heart rate (HR). Respiration rate (RR). Breathing amplitude/tidal-volume proxy and derived ventilation proxy (e.g., minute ventilation V_E or alveolar ventilation V_A) derived from IMU/accelerometer and/or other respiration sensors; optionally normalized by a user-specific rest baseline (A_rest). Optional CO₂ output feature (VCO2 proxy) derived from ptcCO₂ and ventilation proxy and/or respiration rate. Oxygen saturation (SpO2/SaO2). Skin temperature. Demographics: age, sex; optional BMI and/or height/weight. Optional interaction features (examples): RRxCO2, HR×CO2, age×HR, age×RR, sex×HR. Optional ventilation-derived CO2 output feature (ptcCO2+ventilation proxy).

In some embodiments, the system computes one or more physiologically motivated intermediate features that relate transcutaneous CO2 dynamics to a user ventilation estimate. These features can improve robustness of RQ estimation across changing breathing patterns (rest, post-meal, exercise) and provide additional claimable differentiation beyond a purely statistical regression.

Estimate a ventilation proxy from wearable sensors, then combine that ventilation proxy with ptcCO2 (or a calibrated CO2 proxy at the sensing interface) to derive a CO2 output feature (a VCO2 proxy). The VCO2 proxy can be used as an input feature to the RQ model and/or to compute RQ via a hybrid physiology-plus-model approach.

Estimate respiration rate (RR) from one or more sources (e.g., PPG-derived respiration, accelerometer/IMU respiration, or other respiration sensors). Estimate breathing depth from an IMU/accelerometer-derived respiratory-motion component (e.g., band-pass filtering in a respiration frequency range and computing an amplitude metric A_now). Perform an optional user-specific baseline calibration at rest to store A_rest, then compute a normalized depth proxy (e.g., A_now/A_rest) that is less sensitive to strap fit and sensor coupling.

Map RR and the normalized depth proxy (and optionally demographics such as age/sex/height) to a ventilation-related feature (e.g., V_E or V_A) using a calibration mapping and/or a trained model. Apply context gating and quality weighting so ventilation-derived features are suppressed or down weighted during poor contact/high motion; optionally blend the normalized depth proxy toward 1.0 outside of contexts consistent with elevated ventilation. Combine ptcCO₂ with the ventilation-related feature to derive a VCO2 proxy (for example, via proportionality or a learned mapping). Exact constants, windowing, and mapping parameters may be treated as proprietary while still disclosing the feature family and inputs.

Outputs and post-processing: RQ (instantaneous or smoothed) with an estimated confidence score. Derived fuel mix: fat % vs carb % (mapped from RQ range). Metabolic zone classification: fat-dominant, mixed, carb-dominant; optional anaerobic flag when RQ>1. Trend indicators over rolling windows (e.g., 5-60 minutes).

Fuel Utilization Derived from RQ

| | | | Fuel Utilization | |
|---|---|---|---|---|
| RQ | Fat % | Carbohydrate % | Zone | Display Notes |
| <0.70 | 100* | 0* | Below range | Percentages clamped; |
| | | | | show “Below range” |
| 0.70 | 100 | 0 | Fat dominant | Normal display |
| 0.75 | 83 | 17 | Fat dominant | Normal display |
| 0.80 | 67 | 33 | Mixed | Normal display |
| 0.85 | 50 | 50 | Mixed | Normal display |

| | | | Fuel Utilization | |
|---|---|---|---|---|
| RQ | Fat % | Carbohydrate % | Zone | Display Notes |
| 0.90 | 33 | 67 | Carb dominant | Normal display |
| 0.95 | 17 | 83 | Carb dominant | Normal display |
| 1.00 | 0 | 100 | Carb dominant | Normal display |
| >1.00 | 0* | 100* | High intensity/ | Percentages |
| | | | carb overflow | clamped; show |
| | | | | state |

*For RQ values outside 0.70-1.00, percentages are display-clamped for Ul stability and paired with a state label, not interpreted as literal substrate oxidation.

One-time calibration and baseline capture. In some embodiments, the system performs a one-time (or occasional) calibration session before presenting RQ-derived outputs to the user. The calibration establishes user-specific baselines used for normalization, drift checks, and guardrails. Non-limiting baselines captured during a calibration or true-rest session may include Rest breathing-amplitude baseline (A_rest): a user-specific baseline respiration amplitude or tidal-volume proxy captured during a low-motion true-rest period, optionally guided by on-screen prompts. Optional baseline transcutaneous CO₂ level (ptcCO₂_rest): a baseline CO₂ estimate at rest used for later drift checks and plausibility checks. Optional resting energy expenditure baseline (REE_user): a baseline EE estimate derived from true-rest windows and used for normalization of burn-rate displays (e.g., “burn multiple”). Optional sensor/optics baselines: reference-channel levels used to detect optical fouling, LED aging, or ambient leakage.

Calibration updates can be gated by data-quality checks (seal/contact quality, ambient rejection, motion level) so that baselines are only captured or refreshed when the sensing interface is stable. Breath-amplitude gating and context-aware substitution (A_now/A_rest).

In some embodiments, the system uses a breath-amplitude or breathing-depth proxy to improve estimation of CO₂ production features (e.g., a VCO2 proxy) from ptcCO₂. Because depth proxies can be corrupted by voluntary deep breaths, speech, cough, poor strap coupling, or motion artifact, the system can limit how much the depth proxy influences the computation using context-aware gating.

Non-limiting example approach: Compute a raw amplitude ratio ratio_raw=A_now/A_rest (bounded to a plausible range). Compute an exertion/context score from one or more of: heart rate (HR), respiration rate (RR), and motion/activity features (IMU). Blend ratio_raw toward 1.0 when exertion is low (so depth has little or no effect) and allow greater influence when exertion is high. Optionally use age-adjusted or individualized HR thresholds for gating, so that the same physiological intent is recognized across age groups and fitness levels.

If amplitude is missing or fails quality checks, default to ratio_raw=1.0 (no depth adjustment) and/or down weight the affected epoch in the RQ model. Calibration mapping, stabilization, and output guardrails (RQ_raw→RQ_user) In some embodiments, the internal computation produces an intermediate RQ estimate (e.g., RQ_raw) that is subsequently calibrated and stabilized for user presentation (e.g., RQ_user or RQ_cal). This post-processing can improve usability and reduce flicker near user-visible boundaries, while maintaining scientific honesty in labeling.

Non-limiting examples of post-processing steps include Apply a calibration mapping learned from reference data (e.g., a monotonic mapping, linear mapping, or piecewise mapping) to map RQ_raw into a user-facing RQ scale. True-rest stabilization: in detected true-rest conditions, apply hysteresis and/or snapping near a user-visible boundary (e.g., around RQ=0.70) to avoid distracting UI flips caused by noise. Range limiting: constrain displayed RQ to a plausible band and, when outside the nominal range, present “below range” or “above range” labels rather than implying literal 100% fat/carbohydrate oxidation.

Provide confidence/quality indicators and reason codes to the UI (e.g., low contact, high motion, warm-up/stabilizing) and optionally suppress or freeze RQ updates when confidence is low.

Energy expenditure estimation model (inputs, not coefficients). EE is a co-equal primary output. Computing EE via a trained multivariable regression or ML model. The model may use RQ as an input or may compute EE independently from overlapping inputs. In some embodiments, RQ and EE are computed without any direct O₂ measurement, using CO₂-derived features and other physiological context signals alone.

Example EE model inputs (from current modeling work): Sex and anthropometrics (height, BMI; optional weight). Heart rate (HR). Oxygen saturation (SpO2/SaO2). CO2 estimate (ptcCO2) and/or related CO2 features. Optional ventilation proxy (V_E/V_A) and/or CO2 output proxy (VCO2) derived from ptcCO2 and respiration/IMU features. Temperature. Optional interaction features (examples): CO2×HR and CO2×temperature.

EE outputs: Resting energy expenditure (REE) estimate (optional). Activity energy expenditure (AEE) estimate (optional). Total energy expenditure (TEE) over time windows (optional). Cumulative calories burned over day/week/month (optional). Derived features, interactions, and normalization. In some embodiments, the EE computation uses derived features formed from raw inputs. Derived features can improve accuracy, capture physiological interactions, and increase robustness to confounding.

Non-limiting examples include Interaction features that couple CO2 level with heart rate (e.g., CO2×HR), capturing that the effect of HR on EE can depend on CO2 and vice versa. Interaction features that couple CO2 level with temperature (e.g., CO2×temperature), capturing temperature-dependent physiology and sensor behavior. Normalization of burn rate to a user baseline (e.g., REE_user) to form a dimensionless “burn multiple” suitable for intuitive UI display. In some embodiments, REE_user is estimated or updated slowly from true-rest windows using confidence gating, so that the baseline is stable and personalized.

Training-time robustness measures (influence/outlier handling). During model development, the training pipeline may include influence diagnostics and robustness steps to improve generalization. For example, the developer may compute an influence metric (e.g., Cook's distance or related leverage/residual metrics) and optionally trim highly influential outliers before finalizing model parameters. Such trimming is an internal training step; the deployed model can remain a fixed function executed on-device and/or offloaded.

Deployment guardrails, unit checks, and debug outputs. In some embodiments, the EE computation includes guardrails and validation layers that reduce the risk of implausible outputs due to transient sensor artifacts, unit mismatches, or sign mistakes when implementing derived features.

Non-limiting examples include Unit sanity checks on required inputs (e.g., confirming expected units for temperature, CO2, HR, SpO2) and rejecting or down weighting epochs that fail checks. Consistency checks for derived features (e.g., verifying sign conventions for interaction terms and validating ranges of computed CO2×HR and CO2×temperature features). Range limiting and labeling: constraining displayed EE to a plausible band and providing “low confidence” or “data quality” states rather than presenting misleading numbers. Optional debug/telemetry fields that can be logged (locally or to a companion app) to support troubleshooting and model iteration, such as EE_raw, REE_user, burn multiple, confidence score, and reason codes.

Optional derived metrics and coaching. In addition to primary outputs (RQ and EE), the system can derive secondary metrics and generate feedback. These are useful for dependent claims and product differentiation. Fuel utilization % estimates (fat vs carbohydrate) derived from RQ. Time-in-zone metrics (e.g., time in fat-burning zone, time in ketosis-like zone). Meal/workout correlation and post-event recovery tracking (trend-based). Alerts when data quality is low (poor seal/contact, high motion). Personalized targets and recommendations (optional; can be cloud-based).

User experience (watch+app). The system presents RQ and EE in a way that is understandable to users. The UI can be on the host smartwatch (watch face/complication) and/or in a companion app. Example UI concepts (from UI deck).

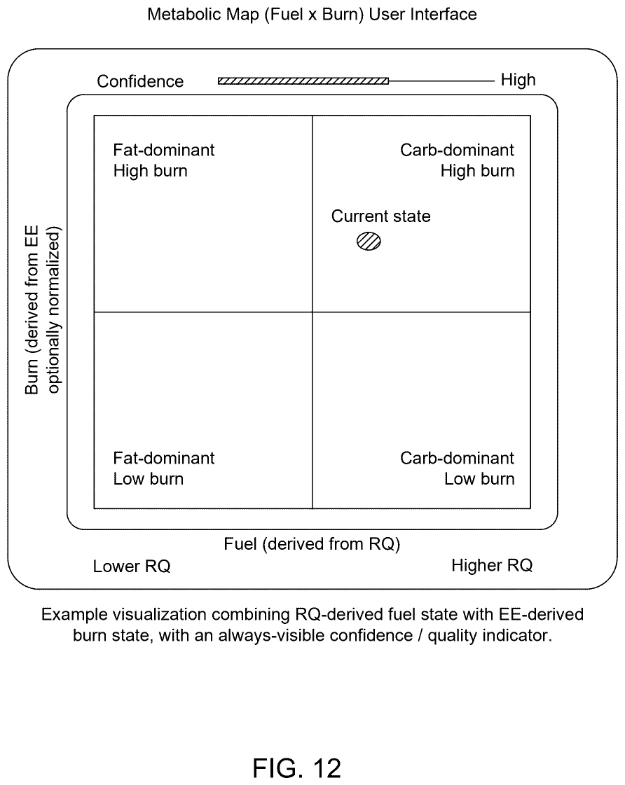

FIG. 12 shows example user interface layouts including (i) a watch/phone display of RQ and fuel split, and (ii) an example “metabolic map” visualization that combines an RQ-derived fuel metric with an EE-derived burn metric, optionally with trend and confidence indicators.

UI features: Simultaneous display of RQ and EE as primary metrics. Fuel bar/slider showing fat vs carbohydrate contribution mapped from RQ. Trend arrow and short-label status (e.g., ‘trending up’, ‘at goal’). Time-in-state tracking (e.g., ketosis duration). Session mode (fasting window, workout, post-meal tracking) with start/stop controls. Confidence indicator and data-quality alerts (e.g., poor contact). User interfaces, operating modes, and example applications

User interface destinations. The system may present outputs on one or more of: On-device display (host smartwatch and/or band display): Minimal readouts such as current RQ, current EE (or kcal/day), and confidence; optional trends.

Companion application: Real-time graphs of RQ and EE, historical trends, event markers (meals/workouts), and alerts. Cloud integration (optional): Secure storage, long-term analytics, sharing with coaches or clinicians, and optional integration with healthcare systems (implementation-dependent and jurisdiction-dependent).

Power management and measurement modes. Because the sensing module uses optical excitation and timed sampling, measurement may be configured as: Continuous monitoring mode: periodic t-DLR cycles (e.g., every N seconds/minutes) with smoothing and confidence scoring.

On-demand mode: user initiates a measurement session (e.g., “check metabolic state”) and the device increases duty cycle briefly. Adaptive duty cycling: the system automatically increases or decreases duty cycle based on contact quality, motion level, user settings, and battery state.

Example deployment configurations. The system may be deployed as: Replacement strap for a host smartwatch. Add-on module attached to an existing strap. Standalone wearable (watch body, patch, armband, etc.) in other embodiments.

Example application domains. The same underlying sensing and modeling pipeline may be configured for: Consumer fitness/wellness emphasizes RQ+EE trends, workout context, and coaching prompts. Athlete/performance/research emphasizes higher sampling rates, raw-feature export, and integration with reference systems for calibration studies. Clinical or supervised settings (optional embodiment): emphasizes higher confidence gating, logging, alerts, and interoperability requirements (implementation-dependent).

Example context-aware interpretation scenarios (RQ+EE+context). These examples illustrate why context matters: the same RQ value can have different interpretations depending on activity level, heart rate, time of day, and signal quality. In these examples, the system computes (i) RQ, (ii) EE, and (iii) a confidence score, and then selects a user-facing interpretation. Scenario A: Rest vs high-intensity differentiation when RQ is high. Observation: RQ rises above a high threshold (example: >1.0). Context A (exercise): HR elevated and motion intensity high→interpret as high-intensity carbohydrate utilization; label as “high intensity/carb-dominant.”. Context B (rest): HR near resting and motion low→interpret as carbohydrate-dominant at rest (e.g., post-prandial); optionally prompt light movement depending on user goals.

EE tie-in: show high EE during exercise case vs low EE during rest case to reinforce interpretation. Scenario B: Low RQ trend during sedentary period. Observation: RQ trending downward over time. Context: motion/steps low for a prolonged period, HR stable→interpret as sedentary period with shift toward fat oxidation; optionally suggest movement depending on settings. EE tie-in: low AEE supports “sedentary” context. Scenario C: Post-meal metabolic response. Observation: RQ increases following a logged meal time or detected pattern.

Context: motion low and HR near resting. Interpret as post-prandial carbohydrate utilization; optionally provide a gentle prompt aligned to goals. EE tie-in: modest EE at rest helps separate meal response from workout response. Scenario D: Post-workout recovery state vs prolonged low intake (avoid diagnosing). Observation: RQ becomes low (example: <~0.7) for a sustained interval. Context A (recent workout): activity detected recently, HR recovery present. Interpret as post-exercise shift toward fat oxidation. Context B (no activity, prolonged interval): low activity+prolonged low RQ+low EE. Interpret as sustained low substrate availability; optionally prompt “check-in” (user-confirmed input such as “fasting/keto/low intake”).

Scenario E: Ketogenic/fasting goal mode (optional user setting). Observation: sustained low RQ with stable activity context. User setting: user indicates ketogenic diet or fasting goal. Action: UI confirms “in target zone” and logs time-in-zone; alerts only if confidence drops or values deviate.

Scenario F: Confidence-aware display behavior. Observation: motion high or contact quality low (e.g., ambient leakage, abnormal ratios). Action: system reduces confidence, suppresses coaching prompts, and displays a “low confidence” indicator or switches to reduced-capability mode until signal quality recovers.

UX/UI specification highlights. A separate UX/UI specification sheet (v7) provides product-facing design guidance. For patent drafting purposes, the following UI concepts can be treated as optional embodiments that support dependent claims and broaden disclosure. The disclosure should remain brand-agnostic (e.g., “host smartwatch”) and should avoid locking into a single visual design.

Information hierarchy (Fuel, Burn, Trust, Meaning). In some embodiments, the interface is organized around a consistent hierarchy to reduce user confusion: Fuel: the user's metabolic fuel mix derived from RQ (e.g., fat-dominant, mixed, carb-dominant), shown as the primary (“hero”) element. Burn: the user's current energy flow derived from EE, used to contextualize the fuel mix (e.g., low burn at rest vs high burn during activity). Trust: a confidence/quality indicator that is always visible and user-actionable (e.g., High/Medium/Low confidence with reason codes). Meaning: a short interpretation statement derived from Fuel+Burn+context (time of day, activity, trends), optionally mode-aware. Three-layer UX architecture (glance, now, deep).

In some embodiments, the system uses a layered UX architecture: Glance layer: watch face/complications showing a minimal subset (e.g., current RQ zone, burn indicator, confidence). Now layer: a primary “Now” screen presenting Fuel+Burn+Trust+a brief interpretation sentence. Deep layer: companion app views (e.g., daily timeline) with trends, event markers (meals/workouts), and explanatory breakdowns. Metabolic Map (Fuel×Burn fusion object).

In some embodiments, the UI presents an optional fusion visualization that combines RQ and EE into a single object (e.g., a 2D crosshair and dot map): X-axis (Fuel): derived from RQ within a nominal display range (e.g., ~0.70 to ~1.00), with out-of-range values pinned and labeled (e.g., “below range (fat-dominant)” or “high intensity/overflow”). Y-axis (Burn): derived from EE and optionally normalized to a user baseline (e.g., REE_user) to form a dimensionless burn multiple (e.g., AEE/REE_user) suitable for intuitive comparison across users and across days.

Crosshair thresholds: horizontal/vertical thresholds can be fixed, user-configurable, and/or mode-dependent (fasting/training/recovery), and can be used to define quadrants such as low-burn vs high-burn and fat-dominant vs carb-dominant. Trust overlay: the confidence/quality indicator can control dot opacity, color/shape, or display suppression to prevent over-interpretation when signal quality is poor.

Confidence-aware UI states and remediation cues. In some embodiments, confidence is not only displayed but also used to drive UI state: Low-confidence state: suppress or de-emphasize RQ/EE numbers and show actionable prompts (e.g., adjust strap fit, remain still briefly, clean/replace consumable). Band-off or incomplete state: detect when the sensing module is not in contact and show a clear “band not measuring” state rather than stale values.

Mode system (goal-aware interpretation without changing raw measurements). In some embodiments, the UI and interpretation layer supports user-selectable or automatically inferred modes, such as fasting, training, recovery, or a performance test. Modes can adjust smoothing, thresholds, labels, and coaching language while leaving the underlying sensor measurements and primary model outputs intact.

FIGS. 1 and 8 show non-limiting wearable form factors including a band/strap embodiment and a smartwatch embodiment; other embodiments include sidecar modules, patches, and armbands.

FIGS. 9 and 10 show non-limiting sensing interface cross-sections, including membrane+sensing element(s)+optics, and illustrate example gas diffusion and optical excitation/emission paths compatible with t-DLR and other optical readouts.

Additional user interface and system architecture examples are shown throughout FIGS. 1-12; the particular layouts and form factors are illustrative and not limiting.

### Example

### Use Case Scenarios for RQ Data.

This example explains how our wearable technology would present RQ data to the end user as well as RQ applications for the end user. These scenarios highlight why trend analysis and context are so critical—because an RQ of 1.1 at rest is very different from an RQ of 1.1 during intense physical exertion. By factoring in activity (via pedometer/accelerometer) and time of day, the wearable can interpret whether the user is overeating, under-eating, vigorously exercising, or in a sedentary state.

Morning RQ Check: Identifying Starvation vs. Normal Fasting.

Scenario: A user wakes up and the wearable senses a low RQ (<0.7).

### Potential Interpretations:

They are in a true starved state and need to eat breakfast.

They are coming off an overnight fast and are still in normal “fat-burning” mode.

### Additional Data:

The wearable detects 8+ hours of inactivity (sleep) via pedometer/accelerometer.

It is early morning (time of day data).

### Action:

The wearable displays a yellow or red indicator (depending on how low the RQ is and how long it has been low).

Prompts the user: “You may be running on empty—consider breakfast to replenish energy.”

By combining RQ with time of day and inactivity cues, the device can distinguish between healthy overnight fasting (mildly low RQ) and a more concerning starved state (prolonged RQ<0.7).

B. Midday RQ Trend: Monitoring Activity vs. Sedentary Behavior.

Scenario: During the workday, the user's wearable continuously tracks RQ and step count.

### Trend Observations:

Over the past few hours, RQ is slowly trending downward from ~0.85 to ~0.73.

Pedometer data shows minimal steps in that same timeframe.

### Potential Interpretation:

The user has been sedentary for an extended period, causing a drift toward fat metabolism (lower RQ).

Action: The wearable displays a yellow indicator and sends a gentle “Time to Move” alert.

It can say, “Your RQ is dropping, and you've been sitting for 2+ hours—try walking for 5-10 minutes.”

If, however, the wearable sees that steps are high and RQ is dropping, it might interpret the situation as a shift from carbohydrate to fat metabolism due to ongoing exercise—thus potentially a green indicator of healthy activity.

C. After Lunch: Detecting Overeating Vs. Normal Metabolic Shift.

Scenario: The user finishes a large meal at noon and remains at their desk.

### Trend Observations:

RQ rises above 1.0 for ~30 minutes, then gradually settles around 0.95.

Pedometer data is low (no movement), heart rate is near resting.

Potential Interpretation: The user ate a large carbohydrate-rich meal (RQ>1.0). The body is predominantly burning carbs at rest.

Action: The wearable might display a yellow indicator for “carb surplus” and provide a gentle nudge: “You've been sedentary after a big meal—walk for 10 minutes to help utilize that energy.”

If, on the other hand, RQ spiked above 1.0 but heart rate and step count were also high, that might indicate intense exercise, where the user is burning carbs quickly. The wearable could then display a green “keep it up!” message.

D. Late Afternoon Drop in RQ: Interpreting Exercise Vs. Undereating.

Scenario: Around 4 PM, the user's RQ starts to drop from ~0.85 down to ~0.68 over an hour.

Data Context: High step count or a recorded workout session ending at 3:30 PM.

### Potential Interpretations:

Post-exercise fat-burning state: The user's body is shifting to fat oxidation.

Prolonged hunger/undereating: The user hasn't eaten anything since lunch, and they're running out of glycogen stores.

### Additional Clues:

A short questionnaire (“How intense was your workout?”/“When did you last eat?”) can confirm context.

Action: If the user confirms “I had a tough run, no snack,” the wearable suggests a high-protein or balanced snack to replenish.

If the user indicates “I haven't eaten at all since noon,” it can recommend a proper meal.

This is a prime example of how a similar RQ (e.g., 0.68) can mean either positive exercise-induced fat burning or an unhealthy undereating scenario—context is everything.

E. Evening RQ Bump: Carbohydrate Replenishment Vs. Overeating.

Scenario: It's dinner time; the user's RQ jumps from ~0.75 to 0.9 within 30 minutes.

Data Context: The wearable sees a medium step count from a moderate walk home.

Mealtime is logged around 6:30 PM.

Potential Interpretations: Normal metabolic shift due to dinner (carbohydrate intake).

Potential overeat if RQ remains elevated (>0.9) for an extended period, and the wearable sees minimal activity.

Action: The device shows a green or yellow indicator based on how long RQ stays high and user's dietary goals. Could say, “Consider light activity after dinner if you want to maintain a balanced metabolism tonight.” F. High RQ (>1.0) During Evening Workout: Positive Training Effect.

Scenario: The user performs high-intensity interval training (HIIT) around 7 PM.

Trend Observations: RQ soars above 1.0 during intervals, heart rate is also elevated, pedometer or accelerometer detects vigorous movement.

Interpretation: This is a normal physiological response to anaerobic or near-anaerobic exercise, indicating heavy carb utilization.

Action: Display a green “Great Job!” or “High Intensity Achieved” message.

Provide post-workout tips: “Stay hydrated, replenish carbs if needed.”

Here, an RQ above 1.0 is not indicative of overeating but rather a sign of intense exertion.

G. Tracking a Low RQ (<0.7) in the Evening: Fasting Vs. Potential Ketosis

Scenario: The user is on a ketogenic diet or intentionally fasting for metabolic reasons.

Trend Observations: RQ is consistently ~0.68-0.69, no major spikes, moderate heart rate if they're gently active.

Interpretation: This may indicate that they are steadily burning fat (and possibly producing ketones).

Action: The wearable could display a green indicator if the user's goal is ketosis, or a yellow if the user is unintentionally under-fueling.

Prompt a user question: “Are you fasting or on a keto diet? If yes, you're on track. If not, consider a balanced meal to avoid energy deficits.”

### H. Overnight Monitoring for Medical or Special Dietary Needs

Scenario: A user with a specific medical condition (e.g., epilepsy on a ketogenic diet) wants to ensure they maintain ketosis overnight.

Data Context: RQ sensor shows ~0.7 or lower sustained through the night.

The device is in a low-power mode but continues to log RQ and minimal movement.

Action: The wearable syncs data each morning to confirm the user stayed in the target RQ zone.

A green indicator shows they maintained ketosis; a yellow if it briefly rose above 0.7, suggesting carb intake or missed dietary protocol.

### I. Real-Time Coaching Prompts and Color Indicators

Below is an example “traffic light” system (red/yellow/green) that the wearable might use, combining RQ values, trends, and context (time of day, steps, heart rate):

### Green

RQ is within an expected zone given the user's activity or dietary goals.

Example: RQ 0.85 while briskly walking, or RQ 1.1 during intervals.

Coaching Prompt: “Keep it up!” or “You're on track.”

### Yellow

RQ slightly mismatched to the user's expected activity or goal.

Example: RQ 1.05 at rest for 2+ hours (possible overeating) or RQ 0.68 with no recent meal (possible undereating).

Coaching Prompt: “Check in: Are you fueling enough?” or “You may be over-consuming carbs.”

### Red

RQ is significantly off target for a prolonged period, or nearing extremes (<0.7 or >1.2) without appropriate context (e.g., intense exercise).

Example: RQ 1.2 at rest for an hour (consistent with very high carb intake but no activity) or RQ<0.7 in the morning with dizziness (possible severe undernutrition).

Coaching Prompt: “Action needed! Please log a meal or consider moderate physical activity.”

### Example Embodiments

A wrist band comprising: a strap configured to be worn around a wrist; and a gas-sensing module coupled to the strap on a user-contact side such that, during wear, the gas-sensing module contacts skin at a measurement site, the gas-sensing module comprising: a gas-permeable membrane configured to be positioned adjacent the skin; a luminescent sensing element responsive to carbon dioxide and optically coupled to the gas-permeable membrane; an excitation light source configured to illuminate the luminescent sensing element; a photodetector configured to detect emission from the luminescent sensing element; and at least one processor operatively coupled to the excitation light source and the photodetector and configured to determine a transcutaneous carbon dioxide metric including partial pressure based on the detected emission.

The wrist band further comprising a luminescent oxygen sensing element configured to provide a transcutaneous oxygen metric or partial pressure.

The wrist band wherein the luminescent sensing element comprises a carbon dioxide-sensitive luminophore and a carbon dioxide-insensitive reference luminophore having a longer effective emission lifetime than the carbon dioxide-sensitive luminophore.

The wrist band wherein the at least one processor is configured to: drive the excitation light source with pulses; compute an ON-window value (A_ON) by integrating or averaging a photodetector signal during a first time window associated with a pulse; compute an OFF-window value (A_OFF) by integrating or averaging the photodetector signal during a second time window after the pulse; and compute a referenced metric based on A_ON and A_OFF to determine the transcutaneous carbon dioxide partial pressure.

The wrist band, wherein the referenced metric comprises a ratio of A_ON to A_OFF or a function derived from the ratio.

The wrist band, wherein the gas-permeable membrane comprises at least one of PTFE, ePTFE, silicone, polyurethane, a fluoropolymer, or a multilayer composite configured to pass gas while inhibiting liquid water.

The wrist band, wherein the luminescent sensing element comprises a removable consumable that comprises the gas-permeable membrane and at least one sensing film or layer, and the wrist band comprises a receptacle configured to position the removable consumable with repeatable alignment relative to the excitation light source and the photodetector.

The wrist band, wherein the removable consumable comprises a membrane-sealed pocket containing particulate or microsphere-based sensing media.

The wrist band, wherein the wrist band is configured as at least one of: (i) a replacement strap for a host smartwatch, or (ii) an add-on strap module, and is configured to exchange data with the host smartwatch and/or a companion device via a wireless interface and/or a wired interface.

The wrist band, wherein the wrist band comprises an opto-mechanical carrier that supports and aligns the excitation light source, an emission filter, the photodetector, and a sensing region of the gas-sensing module, and includes at least one of alignment features, baffles, or seals to reduce ambient light coupling and sweat ingress.

The wrist band, further comprising an analog front end including a transimpedance amplifier and an analog-to-digital converter configured to acquire a time-synchronized photodetector signal relative to the excitation light source pulses.

The wrist band, further comprising at least one computing device including a processor and a user interface, wherein the processor is configured to: receive the transcutaneous carbon dioxide partial pressure from the wrist band; obtain one or more additional physiological or context signals comprising at least one of heart rate, respiration rate, photoplethysmography, peripheral oxygen saturation, skin temperature, barometric pressure, and motion from an inertial measurement unit; and compute respiratory quotient (RQ) and energy expenditure (EE) based at least on the transcutaneous carbon dioxide metric and the one or more additional physiological or context signals.

The wrist band, wherein the processor computes RQ and EE as co-equal primary outputs and provides a measurement-quality or confidence indicator associated with at least one of the outputs.

The system, wherein the processor estimates ventilation as at least one of minute ventilation (V_E) or alveolar ventilation (V_A) using one or more motion-derived features from the inertial measurement unit and at least one of respiration rate or breathing depth, and uses the ventilation estimate to compute a carbon dioxide production metric (VCO₂) from the transcutaneous carbon dioxide partial pressure.

The system, wherein the user interface displays a metabolic map comprising a two-dimensional visualization that combines (i) a fuel axis derived from RQ and (ii) a burn axis derived from EE or a burn-multiple derived from EE.

A method comprising: obtaining, with a wrist band, optical measurements of a luminescent carbon dioxide sensing element through skin at a measurement site; determining a transcutaneous carbon dioxide metric based on the optical measurements; obtaining one or more additional physiological or context signals; computing respiratory quotient (RQ) and energy expenditure (EE) based at least on the transcutaneous carbon dioxide partial pressure and the one or more additional physiological or context signals; and outputting at least one of RQ, EE, or a confidence indicator.

The method above wherein determining the transcutaneous carbon dioxide metric comprises time-domain dual lifetime referencing (t-DLR) including pulsing an excitation light source; integrating or averaging a photodetector signal in an ON window and an OFF window; and computing a referenced metric based on the ON and OFF window values.

The method above wherein computing EE comprises computing EE from estimated VO₂ and VCO₂ using a Weir equation or modified Weir equation.

The method above, further comprising computing at least one of resting energy expenditure (REE), activity energy expenditure (AEE), or total energy expenditure (TEE) based on the computed EE and a user baseline.

The method above, further comprising performing confidence gating by suppressing, flagging, or de-weighting at least one measurement when a contact-quality metric, ambient-light metric, or motion-artifact metric indicates low measurement quality.

### Glossary

A_OFF—Integrated (or averaged) baseline-corrected photodiode signal over the LED-OFF measurement window after a short guard/settle delay (reference-dominated afterglow).

A_ON—Integrated (or averaged) baseline-corrected photodiode signal over the LED-ON measurement window (contains CO₂-sensitive+reference emission).

A_now—Current breath-amplitude metric (derived from respiration/IMU signals) used for ventilation-derived features; often normalized to A_rest.

A_rest—User-specific baseline breath-amplitude metric measured during a guided rest calibration; used to normalize A_now.

ADC—Analog-to-digital converter.

AEE—Activity energy expenditure (calories attributable to activity).

AFE—Analog front end; the analog signal-conditioning electronics between sensor and digital processor (e.g., LED drivers, TIA, filters).

API—Application programming interface.

BLE—Bluetooth Low Energy.

BMI—Body mass index.

CO₂—Carbon dioxide.

EE—Energy expenditure (e.g., kcal/day or kcal/min).

EHR—Electronic health record.

ePTFE—Expanded polytetrafluoroethylene (a common gas-permeable membrane material).

GLM—Generalized linear model.

HPTS—8-hydroxypyrene-1,3,6-trisulfonic acid; a common pH-sensitive fluorophore used in CO₂/pH indicator systems.

HR—Heart rate (beats per minute).

I(t)—Baseline-corrected photodiode signal waveform versus time for a single LED pulse cycle.

IC—Indirect calorimetry (reference method for VO₂/VCO₂ and metabolic measurements).

ID—Identifier (e.g., lot ID, consumable ID).

IMU—Inertial measurement unit (accelerometer/gyroscope).

LED—Light-emitting diode.

LP filter-Long-pass optical filter (e.g., >500 nm cutoff) that blocks blue excitation and passes emission light.

IMU—Inertial measurement unit; typically includes an accelerometer and gyroscope used to estimate motion/activity and, in some embodiments, respiration-related motion.

LR—Luminescence ratio/luminophore ratio metric used in t-DLR (commonly LR=A_ON/A_OFF).

MCU—Microcontroller unit.

ML—Machine learning.

MPH—Miles per hour (used only in example activity descriptions).

NFC—Near-field communication.

OEM—Original equipment manufacturer.

ON/OFF windows—Defined time windows relative to an LED pulse during which measurements are integrated/averaged (e.g., LED-ON window and LED-OFF decay window).

O₂—Oxygen.

PaCO₂—Arterial partial pressure of CO₂ (mmHg); can be used as a reference/training label. Not the same as transcutaneous CO₂, though correlated after calibration.

PPG—Photoplethysmography; optical measurement used for HR and SpO₂ in many wearables.

ptcCO₂—Transcutaneous partial pressure of CO₂ (CO₂ at/through the skin surface as measured by the band's sensor).

QC—Quality control (manufacturing and/or in-field self-test).

QR code—Quick Response code (2D barcode) used for identification/traceability.

REE—Resting energy expenditure.

RFID—Radio-frequency identification.

RQ—Respiratory quotient; ratio VCO2/VO2 and proxy for fuel utilization (fat vs carbohydrate).

RR—Respiration rate (breaths/minute).

Ru—Ruthenium; often refers to ruthenium-based luminophores used as reference and/or

O₂ indicators.

SaO₂—Arterial oxygen saturation (often used interchangeably with SpO₂ in consumer contexts; SaO₂ is typically arterial blood measurement).

SNR—Signal-to-noise ratio.

SpO₂—Peripheral capillary oxygen saturation measured by PPG.

TEE—Total energy expenditure (REE+AEE+other components such as thermic effect of food, depending on definition).

TIA—Transimpedance amplifier; converts photodiode current to voltage.

t-DLR—Time-domain dual lifetime referencing; a referenced optical readout method that uses time windows (not wavelength separation) to separate a short-lifetime indicator from a long-lifetime reference.

UI—User interface.

V_A—Alveolar ventilation (e.g., L/min); in some embodiments estimated as a proxy from RR and breathing depth.

V_E—Minute ventilation (e.g., L/min); in some embodiments estimated as a proxy from RR and breathing depth.

VCO₂—Rate of CO₂ production (e.g., mL/min) used in metabolic calculations.

VO₂—Rate of O₂ consumption (e.g., mL/min) used in metabolic calculations.

PaCO₂—Arterial partial pressure of CO₂ (e.g., mmHg); used as a clinical reference measurement and, in some datasets, a proxy target during model training/validation.

ptcCO₂—Transcutaneous CO₂ (estimated partial pressure at the skin interface); also written PtCO₂ in some literature.

PtCO₂—Transcutaneous CO₂ (synonym of ptcCO₂).

REE_user—A user-specific baseline resting energy expenditure estimate used for normalization (e.g., burn-multiple displays); may be established during true-rest windows and updated slowly over time.

RQ_raw—Intermediate (uncalibrated) respiratory quotient estimate produced by a computational model prior to calibration and guardrails.

RQ_cal/RQ_user—Calibrated/user-facing respiratory quotient output after applying calibration mapping, stabilization (optional), and range limiting/labeling.

UX—User experience.

Burn multiple—A dimensionless burn-rate indicator derived from EE and normalized to a baseline (e.g., AEE/REE_user), suitable for intuitive UI display.

The Weir formula is a formula used in calorimetry, relating metabolic rate to oxygen consumption and carbon dioxide production. In one embodiment, Metabolic rate (kcal per day)=1.440 (3.9 VO2+1.1 VCO2); where VO2 is oxygen consumption in liters per minute and VCO2 is the rate of carbon dioxide production in liters per minute.

A luminescent sensor film is a flexible material that changes its light emission (color, intensity, lifetime) in response to specific analytes or environmental changes, using incorporated luminescent materials like lanthanides, quantum dots, or organic dyes, enabling carbon dioxide monitoring and oxygen sensing and monitoring.

While this disclosure includes specific examples, it will be apparent after an understanding of the disclosure of this application has been attained that various changes in form and details may be made in these examples without departing from the spirit and scope of the claims and their equivalents.

1. A wrist band comprising:
a gas-sensing module comprising a luminescent sensor for transcutaneously measuring carbon dioxide.

2. The wrist band of claim 1 further comprising a luminescent sensor for transcutaneously measuring oxygen.

3. The wrist band of claim 1, wherein the luminescent sensor comprises 8-Hydroxypyrene-1,3,6-trisulfonic acid (HPTS).

4. The wrist band of claim 1, wherein the luminescent sensor comprises a ruthenium or platinum-based dye.

5. The wrist band of claim 1, wherein the luminescent sensor is integrated into the wrist band as an add on strap module, in a smartwatch housing, or as a patch or armband.

6. The wrist band of claim 1, wherein the luminescent sensor comprises a solid sensing disk/pad or a membrane-sealed pocket comprising particulate or microsphere-based sensing media.

7. The wrist band of claim 1, wherein the wrist band is a wrist band for a smart watch.

8. The wrist band of claim 7, wherein the wrist band is configured to attach to a host smartwatch; and
the luminescent sensor is configured to exchange data with the smartwatch via one or more communication interfaces including a wireless and/or a wired interface.

9. The wrist band of claim 1, wherein the luminescent sensor comprises a membrane comprising polytetrafluoroethylene.

10. The wrist band of claim 1, wherein the luminescent sensor comprises:
a gas permeable membrane layer;
a CO₂ sensitive luminescent sensor layer;
an O2 sensitive luminescent sensor layer;
one or more excitation light source layers;
one or more photodetectors; and
an optical window or cavity layer separating the CO2 and O2 luminescent sensor layers from the one or more excitation light source layers and one or more photodetectors.

11. The wrist band of claim 10, wherein the CO₂ luminescent sensor layer includes two luminophores comprising a CO₂ sensitive luminophore, and a CO₂ insensitive reference luminophore.

12. The wrist band of claim 10, wherein the photodetector is a photodiode.

13. A smartwatch comprising:
a wrist band comprising a luminescent sensor for transcutaneously measuring carbon dioxide;
a luminescent sensor for transcutaneously measuring oxygen; and
the luminescent sensors are configured to exchange data with the smartwatch via one or more communication interfaces including a wireless and/or a wired interface.

14. A method of measuring Respiratory Quotient (RQ) comprising:
transcutaneously measuring a partial pressure of carbon dioxide with a wrist band comprising a luminescent sensor for transcutaneously measuring carbon dioxide, to calculate a volume of carbon dioxide based on the measured partial pressure of carbon dioxide;
transcutaneously measuring a partial pressure of oxygen with a wrist band comprising a luminescent sensor for transcutaneously measuring oxygen, to calculate a volume of oxygen based on the measured partial pressure of oxygen; and
calculating a Respiratory Quotient (RQ) by dividing the measured volume of carbon dioxide by the measured volume of oxygen.

15. The method of claim 14, wherein the measuring the volume of carbon dioxide and the measuring the volume of oxygen comprises time-domain dual lifetime referencing (t-DLR).

16. The method of claim 14, wherein the wrist band comprises a smartwatch.

17. The method of claim 14, wherein the luminescent sensors are configured to exchange data with the smartwatch via one or more communication interfaces including a wireless and/or a wired interface.

18. The method of claim 14, further comprising using the calculated Respiratory Quotient (RQ) to calculate Resting Energy Expenditure (REE) and Activity Energy Expenditure (AEE).

19. The method of claim 18, further comprising calculating the sum of Resting Energy Expenditure REE, Activity Energy Expenditure AEE, and a Thermic Effect of Feeding (TEF) to produce a Total Energy Expenditure (TEE).

20. The method of claim 14, further comprising calculating an Energy Expenditure using the Weir equation.
